(19) United States
(12) Patent Application Publication
(10) Pub. No.: US 2003/0008369 A1
(43) Pub. Date: Jan. 9, 2003

(54) Genetic and epigenetic manipulation of ABC transporters and ecto-phosphatases

(75) Inventors: J. Brian Windsor, Austin, TX (US); Stan J. Roux, Austin, TX (US); Alan M. Lloyd, Austin, TX (US)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701-3271, US

(73) Assignee: Board of Regents, The University of Texas System

(21) Appl. No.: 10/134,019

(22) Filed: Apr. 25, 2002

Related U.S. Application Data
(63) Continuation-in-part of application No. 09/261,825, filed on Mar. 3, 1999.
Application No. 09/261,825 is a continuation-in-part of application No. 09/244,792, filed on Feb. 5, 1999.

(51) Int. Cl. C12N 9/12; C12N 9/00

(52) U.S. Cl. 435/194; 435/183

(57) ABSTRACT

The present invention relates to methods for modulating the growth of tumor and pathogen cells and the resistance of cells to foreign compounds, i.e. drugs, antibiotics, etc. by altering the ATP gradient across biological membranes. The altering of the ATP gradient across biological membranes is achieved through the manipulation of ecto-phosphatase activity and ABC transporter molecule activity which may also be useful to confer herbicide resistance to plants, confer antibiotic resistance to bacteria, confer drug resistance to yeast cells, or to reduce resistance in cells to facilitate chemotherapeutic treatments, and to reduce resistance in bacteria and yeast. The present invention is also directed to the methods for identifying ecto-phosphatase inhibitors and uses thereof.

### BACKGROUND OF THE INVENTION

The present application is a continuation-in-part of co-pending U.S. patent application Ser. No. 09/261,825, filed Mar. 3, 1999, which application was a continuation-in-part of U.S. patent application Ser. No. 09/244,792, filed Feb. 5, 1999. The entire text of each of the above-referenced disclosures is specifically incorporated by reference herein without disclaimer. The present invention involves subject matter developed under NSF Grant Numbered IBN9603884 and other federal funds, so that the United. States Government may have certain rights herein.

### FIELD OF THE INVENTION

The present invention relates generally to the fields of cellular biology. More particularly, it concerns methods and compositions for the modulation of cellular resistance to cytotoxic agents.

### DESCRIPTION OF RELATED ART

Transport Processes

Cells can use a phenomenon called symport to move soluble products across biological membranes. Symport is a form of coupled movement of two solutes in the same direction across a membrane by a single carrier. Examples of proton and sodium-linked symport systems are found in nearly all living systems. The energetics of the transport event depend on the relative size and electrical nature of the gradient of solutes.

Transport processes have been classified on the basis of their energy-coupling mechanisms. Currently there are four classifications: (1) Primary Active Transport which uses either a chemical, light or electrical energy source, (2) Group Translocation which uses chemical energy sources, (3) Secondary Active Transport which uses either a sodium or proton electrochemical gradient energy source, and (4) Facilitated Diffusion which does not require an energy source (Meyers, 1997). The present invention is related to transport molecules belonging to the first class of transport processes, primary active transport, and therefore, this type of transport will be discussed in further detail.

Primary active transport refers to a process whereby a “primary” source of energy is used to drive the active accumulation of a solute into or extrusion of a solute from a cell. Transport proteins include P-type ATPases and ABC-type ATPases, as well as V-type and E-type ATPases. These types of transport systems are found in both eukaryotes and prokaryotes. The bacterial ABC-type transporters, which are ATP-driven solute pumps, have eukaryotic counterparts. Additionally, many transmembrane solute transport proteins exhibit a common structural motif. The proteins in these families consist of units or domains that pass through the membrane six times, each time as an a-helix. This has led to the suggestion that many transport proteins share a common evolutionary origin, but this is not true of several distinct families of transport proteins.

Numerous structurally distinct bacterial permeases, as well as several homologous eukaryotic transport systems, share a common organization (Meyers, 1997). Two hydrophilic domains or proteins function to couple ATP hydrolysis in the cytoplasm to activate substrate uptake or efflux, and two hydrophobic domains or proteins function as the transmembrane substrate channels. These proteins or protein domains constitute what is referred to as the ABC (ATP-binding cassette) superfamily. Either the two hydrophilic domains or proteins or the two hydrophobic domains or proteins (or both) may exist either as heterodimers or homodimers. If, as in most bacterial systems, each of these constituents is a distinct protein, then either two, three, or four genes will code for them, depending on whether both are homodimers, one is a homodimer and one is a heterodimer, or both are heterodimers, respectively. The best characterized of the eukaryotic proteins included in this family are the multidrug-resistance (MDR) transporter and the cystic fibrosis related chloride ion channel of mammalian cells (cystic fibrosis transmembrane conductance regulator or CFTR) (Meyers, 1997).

Multidrug Resistance

Multidrug resistance (MDR) is a general term that refers to the phenotype of cells or microorganisms that exhibit resistance to different, chemically dissimilar, cytotoxic compounds. MDR can develop after sequential or simultaneous exposure to various drugs. MDR can also develop before exposure to many compounds to which a cell or microorganism may be found to be resistant. MDR which develops before exposure is frequently due to a genetic event which causes the altered expression and/or mutation of an ATP-binding cassette (ABC) transporter (Wadkins and Roepe, 1997). This is true for both eukaryotes and prokaryotes.

One prominent member of the ABC family, P-glycoprotein (Pgp; also known as multidrug resistance protein or MDR1), which is a plasma-membrane glycoprotein that confers a multidrug resistance (MDR) phenotype on cells, is of considerable interest because it provides one mechanism of possibly inhibiting resistance in tumor cells to chemotherapeutic agents (Senior et al., 1995). Pgp is a single polypeptide of ˜1280 amino acids with the typical ABC transporter structure profile. Studies have shown that over expression of Pgp is responsible for the ATP-dependent extrusion of a variety of compounds, including chemotherapeutic drugs, from cells (Abraham et al., 1993).

Over one-hundred ABC transporters have been identified in species ranging from Escherichia coli to humans (Higgins, 1995). For example, the bacteria Lactococcus lactis expresses an ABC transporter, LmrA, which mediates antibiotic resistance by extruding amphiphilic compounds from the inner leaflet of the cytoplasmic membrane (Van Veen et al., 1998). Furthermore, over-expression of LmrA can confer MDR in human lung fibroblasts and LmrA has similar molecular and biochemical properties to Pgp. Id. This demonstrates that bacterial LmrA and Pgp are functionally interchangeable. Id. Additionally, the plant Arabidopsis thaliana encodes an ATP transporter, AtPGP-1, which is a putative Pgp homolog (Dudler and Hertig, 1992). Similarly, the yeast Saccharomyces cerevisiae equivalent of Pgp, STS1 (Bissinger and Kucher, 1994) has been cloned and shown to confer multidrug resistance when over-expressed in yeast, as has the yeast Pdr5p (Kolacskowski et al., 1996,). Taken together, these results suggest that this type of multidrug resistance efflux pump is conserved from bacteria to humans.

While various theories of ABC transporter function have become popular, there is still no precise molecular-level description for the mechanism by which over-expression lowers intracellular accumulation of drugs, in particular how Pgp lowers intracellular accumulation of chemotherapeutic drugs. However, it has been shown that Pgp over-expression also changes plasma membrane electrical potential and intracellular pH which could potentially greatly affect the cellular flux of a large number of compounds to which Pgp confers resistance (Wadkins and Roepe, 1997). Also included in the ABC transporter superfamily are the Cystic Fibrosis Transmembrane Conductance Regulator (CFTR) and the Sulfonyl Urea Receptor (SUR). CFTR and SUR are expressed in the lung epithelium and the β cells of the pancreas, respectively, as well as in other tissues. CFTR functions as a low conductance ATP and cyclic AMP-dependent Cl⁻ channel that also appears to have additional important functions, such as modulation of epithelial NA⁺ regulation of outwardly rectified chloride channels (Wadkins and Roepe, 1997).

Mutations in the CFTR gene produce altered CFTR proteins with defects in CFTR function, leading to profound alterations in epithelial salt transport and altered mucous properties in cystic fibrosis patients that result in chronic lung infections associated with the disease. Id. SUR is triggered by sulfonyl urea drugs to depolarize pancreatic P cells that leads to Ca²⁺ influx, which stimulates fusion of insulin containing vesicles to the plasma membrane. Id. An ATP transporter hypothesis has been suggested for Pgp, CFTR and SUR which theorizes that these ABC transporters function as ATP transport channels (Abraham et al., 1993; Schweibert, 1995; Al-Awgati, 1995). The ATP channel hypothesis, however, has been viewed with skepticism. This is partly due to the inability to show the same results with preparations including purified and reconstituted CFTR, suggesting that the ATP conductance that was originally observed may have been mediated by another protein, not present in the purified system, that is influenced by CFTR (Wadkins and Roepe). There has been no such negative data reported with respect to the ATP charnel hypothesis for Pgp or SUR, but the controversy over CFTR has raised doubt for Pgp and SUR as well.

In support of the ATP channel hypothesis (Huang et al., 1992) have suggested that extracellular ATP leads to elevations in pH, and Weiner et al., (1986) have suggested that extracellular ATP may regulate Na⁻/H⁺ exchange in Ehrlich ascites tumor cells. It has also been observed that changes in Pgp levels affects pH and plasma membrane electrical potentials which could be connected to recent observations suggesting the involvement of ATP transport in MDR.

Additionally, Abraham et al., (1993) have reported that the addition of extracellular ATP to MDR cell lines confers sensitivity to drugs abolishing MDR. The data for this effect were not presented in the article and no further explanation was given for this phenomenon. Furthermore, there have been no subsequent publications addressing or explaining this effect.

Furthermore, Ujhazy et al., (1996) have shown that ecto-5′-nucleotidase is up-regulated in certain MDR cell lines. Ecto-5′-nucleotidase is the final enzyme in the extracellular pathway for salvage of adenosine from phosphorylated purines (Zimmerman, 1992). The proposed hypothesis for the involvement of ecto-5′-nucleotidase in drug resistance considers its role in the maintenance of intracellular ATP pools through the adenosine salvage pathway (Ujhazy et al., 1996). Ecto-5′-nucleotidase specifically acts in adenosine salvage pathways, converting AMP to adenosine which is more readily taken up by the cell and utilized as a precursor for ATP production. Therefore, ecto-5′-nucleotidase may be acting in certain MDR cell lines as a mechanism by which the cell circumvents the loss of ATP (due to up-regulated transport proteins which possibly form ATP transport channels) by creating higher levels of adenosine from which the cell can produce ATP. Correspondingly, 63% of MDR cell line variants tested expressed ecto-5′- nucleotidase. These observations suggested that a salvage mechanism for extracellular nucleotides may be another way by which certain MDR cells counterbalance their ATP losses from efflux induced by the over-expression of ABC transporters involved in MMR. Consistent with this hypothesis, inhibitors of ecto-5′-nucleotidase conferred sensitivity to certain drugs in MDR cell lines which over-express the ecto-5′-nucleotidase.

It is also interesting to note that yeast, which do not have an adenosine salvage pathway (Boyum and Guidotti, 1997), do contain a PGP-like gene called STS 1 (Bissinger and Kucher, 1994). Therefore, since the adenosine salvage pathway is unlikely to be involved in yeast multidrug resistance, other mechanisms are likely to exist.

Recent reports have confirmed the existence of ATT in the extracellular matrix (ECM) of both multicellular organisms and unicellular organisms (Sedaa et al., 1990; Boyum and Guidotti, 1997), respectively. However, no such reports are available which suggest the existence of ATP in the ECM of plants before the present invention. These reports have prompted further investigations of the fate of ATP outside the cell. One of the largest gradients in biological systems is that of ATP. It is a million-fold more concentrated inside the cell than outside. Apyrases are enzymes whose unifying characteristic is their ability to hydrolyze the gamma phosphate of ATP and to a lesser extent, the beta phosphate of ADP (Plesner, 1995). Most apyrases are expressed as plasma membrane associated proteins with their hydrolytic activity facing into the ECM (Wang and Guidotti, 1996). Extracellular apyrases are generally referred to as ecto-apyrases. Given reports that show the existence of extracellular ATP, one observation regarding ectoapyrase is that it hydrolyzes the extracellular ATP. In fact, work in animal systems has shown that apyrases hydrolyze ATP in the ECM as part of the adenosine salvage pathway conjointly with ecto-5′ ectonucleotidase (Che, 1992). The existence of a similar ecto-apyrase system has not been reported in plants prior to the present invention. Additionally, ecto-apyrases have not been shown, prior to the present invention, to have a role in MDR.

While some references appear to indicate that MDR may act at the level of ATP transport, the role of ATP in MDR has not been adequately elucidated and has remained a point of contention in the field. The present invention provides insight into the role of ATP transport in MDR by showing that the extracellular ATP pool in cells is critical in MDR. While the adenosine salvage pathway may help compensate for ATP losses in MDR by providing a mechanism to recoup adenosine, it is not the critical aspect of the role of ATP in MDR as evidenced by the observation that only a subset of MDR cell lines resort to this mechanism via the up-regulation of ecto-5′-nucleotidase to maintain drug resistance. In fact, the previous data teach away from modulating extracelluar ATP levels and place the focus on mechanisms which are involved in modulating intracellular ATP levels. Since AMP is the preferred substrate for ecto-5′-nucleotidase, with ATP and ADP being poor substrates (Zimmerman, 1992), it is unlikely that ecto-5′-nucleotidase is involved in modulating extracellular levels of ATP. While high levels of ATP have been demonstrated to be useful in the inhibition of tumor growth, its effects on tumor cells have been shown to prevent cell growth and induce cell death through the inhibition of the S phase of the cell cycle. U.S. Pat. No. 4,880,918. There has been no implication, prior to the present invention, of the importance of modulating extracellular ATP levels in MDR.

Additionally, there has been no identification of specific inhibitors of apyrase (an ecto-phosphatase). Such inhibitors and methods for identifying such inhibitors would be useful for studying the importance of ecto-phosphatases in MDR, for modulating MDR and in industrial applications (e.g. determining the titer of microbia in soil).

It would be particularly useful to have more effective mechanisms by which to modulate drug resistance in various organisms. In particular, since the use of Pgp inhibitors has not been totally efficient in overcoming the resistance seen in tumor cells which have been repeatedly exposed to chemotherapeutic agents, it would be useful to have other mechanisms by which to combat such resistance in tumor cells to provide more effective chemotherapeutic treatments. Furthermore, there are many other applications for the modulation of drug resistance which are contemplated by the present invention, such as the engineering of herbicide resistant plants for use in agriculture.

### SUMMARY OF THE INVENTION

The present invention is directed to a method for the modulation of drug resistance in cells. In one embodiment, resistance is conferred through over-expression by genetic manipulation of ABC transporters and ecto-phosphatases which are capable of affecting extracellular ATP pools and thus affecting the ATP gradient across biological membranes. Conference of resistance is useful to achieve herbicide resistance in plants, drug resistance in yeast (i.e. resistance to anti-fungal agents) in biotechnology applications, antibiotic resistance in bacteria in biotechnology applications and for drug resistance in eukaryotic tissue culture cells in biotechnology applications. In another embodiment, loss of drug resistance is achieved by suppressing the breakdown of extracellular ATP through the down-regulation of ectophosphatases in the presence or absence of the down-regulation of ABC transporters. Loss of resistance is useful to mitigate drug resistance problems associated with chemotherapy and in the treatment of infections from resistant strains of microorganisms. The modulation of drug resistance is achieved, at least in part, by altering the ATP gradient across biological membranes through the aforementioned manipulation of ABC transporters and ecto-phosphatases. The present invention is also directed to methods for the identification of inhibitors of ecto-phosphatases and uses thereof.

One aspect of the invention provides a method of inhibiting the growth of a bacterial cell comprising contacting the cell with an ectophosphatase inhibitor, which includes an apyrase inhibitor. In one embodiment of the invention, the ectophosphatase inhibitor is represented by formula XV or a pharmaceutical salt thereof. In the method, the cell may be resistant to at least a first antibiotic, including, for example, methicillin. Such a cell may comprise an upregulated ecto-phosphatase relative to a bacteria that is not resistant to said antibiotic. The method of the invention may be further defined as a method of increasing the effectiveness of an antibiotic and further comprise contacting the cell with at least a second compound capable of inhibiting the growth of the cell. In certain embodiments of the invention, the compound of formula XV may be comprised in a physiologically acceptable carrier or diluent. The step of contacting may comprise, for example, administering said composition to a mammal infected with said cell, including a human. The bacterial cell may be any type of cell, including, a Staphylococcus. In one embodiment of the invention, the cell is a Staphylococcus aureus cell.

Another aspect of the current invention provides a method for inhibiting the growth a tumor cell comprising contacting said cell with an ectophosphatase inhibitor. In one embodiment of the invention the ectophosphatase inhibitor is represented by formula VI, VIII or X or is a pharmaceutically acceptable salt thereof. The cell may be resistant to at least a first chemotherapeutic agent, including, for example, vinblastine. Such a cell may comprise an upregulated ecto-phosphatase relative to a tumor cell that is not resistant to said first chemotherapeutic agent. The method may, in certain embodiments, further comprise contacting said cell with at least a second compound capable of inhibiting the growth of the cell. In one embodiment of the invention, the compound of formula XV is in a composition comprising a physiologically acceptable carrier or diluent. The step of contacting may comprise administering said composition to a mammal with cancer, including a human. In certain embodiments of the invention, the cell is a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a bone cancer cell, a bone marrow cancer cell or a soft tissue cancer cell.

Yet another aspect of the invention provides pharmaceutical compositions comprising an ectophosphatase inhibitor, including the compounds of formulae I-XX. Such compositions may further comprise and antibiotic or chemotherapeutic. The ectophosphatase inhibitor may increase the effectiveness of such agents. The inhibitors may further increase the accessibility of certain drug molecules, including chemotherapeutics and antibiotics, to parts of the body in which it is otherwise difficult to achieve therapeutic levels of the drug, for example, due to the efflux of the drug across a membrane. For example, the blood-brain barrier may be made more permeable by use of an ectophosphatase inhibitor in accordance with the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C Expression of apyrase in pea and in transgenic plants (FIG. 1A) Immunoblot analysis of subcellular fractions from etiolated pea plants. (FIG. 1B) Top, the total phosphate accumulated in the shoots of three independent transgenic plants. Bottom, a corresponding Immunoblot performed on protein from ECM of wild-type and transgenic plants. (FIG. 1C) Assay of phosphatase activity in the ECM fraction of OE1 and wild-type.

FIGS. 2A-C Transport of the products of ATP hydrolysis by transgenic plants overexpressing apyrase and by wild-type plants.

FIGS. 3A-D Conference of resistance to cycloheximide (FIG. 3A, B) and nigencin (FIG. 3C, D) in wild-type and ecto-phosphatase deficient yeast over-expressing the Arabidopsis plant ABC transporter, AtPGP-1.

FIGS. 4A-B-3 Conference of resistance to cycloheximide (FIG. 4A) and cytokinin (FIG. 4B-1-FIG. 4B-3) in Arabidopsis plants over-expressing either the ecto-phosphatase, apyrase, or the ABC transporter, AtPGP-1.

FIGS. 5A-B Graph showing, the growth turbidity of YM4 yeast over-expressing the Arabidopsis plant ABC transporter AtPGP-1 grown in cycloheximide (FIG. 5A) or nigericin (FIG. 5B).

FIG. 6 Graph showing germination rate of Arabidopsis plants grown in the presence of cycloheximide which over-express either the ecto-phosphatase, apyrase, or the ABC transporter AtPGP-1.

FIG. 7 Graph of steady-state levels of ATP in the extracellular fluid of wildtype yeast cells grown in the presence or absence of glucose and in the presence or absence of over-expression of the Arabidopsis plant ABC transporter, AtPGP-1.

FIG. 8 Graph showing that over-expression of Arabidopsis plant ABC transporter, AtPGP-1, in yeast can double the steady-state levels of ATP in the extracellular fluid.

FIG. 9 Graph showing that a yeast mutant, YMR4, that has a deficient ectophosphatase, accumulates ATP in the extracellular fluid and the over-expression of AtPGP-1 increases the accumulation of ATP.

FIG. 10 Graph showing results of a pulse-chase experiment in either wildtype yeast cells or a yeast mutant, YMk4, which is deficient in ecto-phosphatase activity, in the presence and absence of over-expression of Arabidopsis plant ABC transporter, AtPGP-1, demonstrating an early differential ATP efflux of cells over-expressing AtPGP-1.

FIG. 11 Graph of ATP levels on the surface of leaves of Arabidopsis plants over-expressing AtPGP-1 (MDR 1).

FIG. 12 Effects of phosphatase inhibitor in wild-type and AtPGP-1 (MDR1) overexpressing Arabidopsis plants.

FIG. 13 Growth effects of cycloheximide and extracellular ATP on wildtype and MDR1 overexpressing S. cerevisiae yeast cells which have either never seen cycloheximide or which have been previously selected in cycloheximide.

FIG. 14 Growth effects of cycloheximide, adenosine and phosphate on wildtype and AtPGP-1 overexpressing S. cerevisiae yeast cells.

### DETAILED DESCRIPTION OF THE INVENTION

The present invention is concerned with modulating the drug resistance pathways of cells in order to either confer or overcome resistance to certain drug molecules. Such modulation entails modulation of an extra-cellular phosphatase (ecto-phosphatase) and an ABC (ATP-binding cassette) transporter in order to achieve the desired effect on drug resistance. Stimulation of the ecto-phosphatase either alone or together with stimulation of the ABC transporter yields an increased resistance to drug molecules while inhibition of the ectophosphatase alone or together with the ABC transporter yields reduced resistance to the drug molecule. Drug resistance is achieved through the altering of the ATP gradient across biological membranes which is effectuated through the modulation of an ecto-phosphatase either alone or together with an ABC transporter molecule. Modulation of drug resistance as described herein is useful in conferring herbicide resistance to plants; conferring drug resistance to microorganisms and tissue culture cells; reducing drug resistance in tumor cells for improved chemotherapy applications; and reducing resistance to antibiotics, antifungal agents, and other drugs in microorganisms for the treatment of infections and disease. The present invention is also directed to methods for identifying inhibitors of ecto-phosphatases and uses thereof.

For purposes of clarity of description, and not by way of limitation, the detailed description of the invention is divided into the following subsections:

(i) conference of herbicide resistance in plants;

(ii) conference of drug resistance in recombinant research applications;

(iii) inhibition of drug resistance in chemotherapy;

(iv) inhibition of drug resistance in microorganisms to treat infection; (v) ecto-phosphatase inhibition

Conference of herbicide resistance in plants

The present invention is directed to a method for the modulation of drug resistance in plants, particularly herbicide resistance, in part through the manipulation of the ATP gradient across biological membranes. In accordance with the invention, the manipulation of extracellular ATP levels and hence the ATP gradient across biological membranes in plant cells by the over-expression of a 1VIDR-ABC transporter and an ecto-phosphatase, results in resistance to certain plant hormones, drugs and herbicides. Such resistance is useful in horticulture of recombinant crops for the elimination of other unwanted plants (e.g. weeds) which are not resistant. The invention is based, in part, on the unexpected observation that the over-expression of either an ecto-phosphatase, or an ABC transporter can confer resistance to certain drugs and herbicides in plants.

Up-regulation as used herein refers to increasing the activity of a molecule within a cell by either providing an outside source of the molecule (e.g. an expression cassette containing a DNA encoding the molecule) either in single copy or multiple copies which when expressed in the cell increases the amount of the molecule in the cell, by increasing the transcription of the endogenous or exogenous molecule to increase the amount of the molecule in the cell, or by modifying the exogenous or endogenous molecule in the cell post-translationally to achieve an increase in activity of the molecule. Down-regulation as used herein refers to decreasing the activity of a molecule in a cell by either decreasing the amount of the molecule in the cell (this may be achieved by over-expression of an anti-sense RNA corresponding to the molecule or by inhibiting factors necessary for the expression of the molecule) or by modifying the exogenous or endogenous molecule in the cell post-translationally to achieve a decrease in activity. Such post translational modifications may include phosphorylation, adenylation, glycosylation, ubiquitinylation, acetylation, methylation, farnesylation, myristilation and sulfation.

The ecto-phosphatases remove phosphate from any ATP extruded from the cell, removing the gradient potentiating efflux of drugs from the cell.. Ecto-phosphatases as referred to herein do not include extracellular phosphatases involved in the adenosine salvage pathway. NIDR ABC transporters form channels which facilitate the efflux of molecules, including drugs, from cells. This efflux is likely effectuated through the “piggy-back” efflux of drug molecules with ATP, a phenomenon known as symport.

In one embodiment of the invention, the over-expression of an ecto-phosphatase confers drug resistance in both wild-type and/or genetically engineered plants. This effect is seen in plant cells over-expressing plant apyrase grown in the presence of (1) cycloheximide, a potent inhibitor of protein expression, (2) nigericin, an antibiotic which effects ion transport, and (3) N₆ (2-isopentenyl) adenine, a cytokinin plant hormone which is herbicidal at micromolar and millimolar concentrations.

In another embodiment of the invention, the over-expression of an ABC transporter confers drug resistance in wild-type and genetically engineered plants. In a preferred embodiment, the ABC transporter which is over-expressed is the Arabidopsis ABC transporter AtPGP-1. The over-expression of AtPGP-1 can confer resistance in plants to cycloheximide, nigencin and cytokinins.

In a preferred embodiment of the invention the effect of over-expression of both an MDR-ABC transporter and an ecto-phosphatase is enhancement of the ATP gradient across biological membranes and thus stimulation of resistance to certain plant hormones and herbicides. In a particularly preferred embodiment of the invention, the MDR-ABC transporter which is over-expressed is the Arabidopsis AtPGP- 1 and the ecto-phosphatase that is overexpressed is apyrase.

The invention particularly contemplates the conference of resistance in plants to herbicides which resemble established drugs implicated in multidrug resistance, as well as plant hormones such as cytokinin, auxins, gibberellins and brassinosteroids. The present invention also contemplates the conference of resistance in plants to the nonlimiting list of chemicals, such as those set forth in Table 1*.

TABLE 1

| Common Name | Chemical Name |
|---|---|
| acetochlor | -chloro-N-(ethoxymethyl)-N-(2-ethyl-6-methylphenyl)acetamide |
| acifluorfen | 5-[2-chloro-4-(trifluoromethyl)phenoxy]-2-nitrobenzoic acid |
| acrolem | 2-propenal |
| alachlor | 2-chi oro-N-(2,6-diethylphenyl)-N-(methoxymethyl)acetamide |
| allyl alcohol | 2-propen-1-ol |
| ametryn | N-ethyl-N′-(1-methylethyl)-6-(methylthio)-1,3,5-triazine-2,4-di amine |
| amitrole | 1H-1,2,4-triazol-3-amine |
| AMS | ammonium sulfamate |
| arsenic acid | arsenic acid |
| asulam | methyl[(4-am]nophenyl)sulfonyl]carbamate |
| atraton | N-ethyl-6-methoxy-N′-(1-methylethyl)-1,3,5-tri azine-2,4-di amine |
| atrazine | 6-chloro-N-ethyl-N′-(1-methylethyl)-1,3,5-triazine-2,4-diamine |
| azafenidin | 2-[2,4-dichloro-5-(2-propynyloxy)phenyl]-5,6,7,8-tetrahydro-1,2,4- |
| | triazolo[4,3-a]pyndin-3(2H)-one |
| azimsulfuron | N-[[(4,6-dimethoxy-2-pyrimidinyl)amino]carbonyl]-1-methyl-4-(2-methyl- |
| | 2H-tetrazol-5-yl)-1 H-pyrazole-5-sulfonamide |
| barban | 4-chloro-2-butynyl 3-chlorophenylcarbamate |
| BCPC | 1-methylpropyl 3-chlorophenylcarbamate |
| benazolin | 4-chloro-2-oxo-3(2H)-benzothiazoleacetic acid |
| benefin | N-butyl-N-ethyl-2,6-dinitro-4-(trifluoromethyl)benzenamine |
| bensulfuron | 2-[[[[[(4,6-dimethoxy-2- |
| | pyrimidinyl)amino]carbonyl]amino]sulfonyl]methyl]benzoic acid |
| bensulide | 0,0-bis(1-methylethyl)S-[2- |
| | [(phenylsulfonyl)amino]ethyl]phosphorodithioate |
| bentazon | 3-(1-methylethyl)-(1H)-2,1,3-benzothiadiazin-4(3H)-one 2,2-dioxide |
| benzadox | [(benzoylamino)oxy]acetic acid |
| benzipram | 3,5-dimethyl-N-(1-methylethyl)-N-(phenylmethyl)benzamide |
| benzofluor | N-[4-(ethylthio)-2-(trifluoromethyl)phenyl]methanesulfonamide |
| benzoylprop | N-benzoyl-N-(3,4-dichlorophenyl)-DL-alanine |
| benzthiazuron | N-2-benzothiazolyl-N′-methylurea |
| bifenox | methyl 5-(2,4-dichlorophenoxy)-2-nitrobenzoate |
| borax | sodium tetraborate |
| bromacil | 5-bromo-6-methyl-3-(1-methylpropyl)-2,4(1H, 3H)pyrimidinedione |
| bromofenoxim | 3,5-dibromo-4-hydroxybenzaldehyde O-(2,4-dinitrophenyl)oxime |
| bromoxyml | 3,5-dibromo-4-hydroxybenzonitrile |
| butachlor | N-(butoxymethyl)-2-chloro-N-(2,6-diethylphenyl)acetamide |
| butam | 2,2-dimethyl-N-(1-methylethyl)-N-(phenylmethyl)propanamide |
| butamifos | O-ethyl 0-(5-methyl-2-nitrophenyl)1-methylpropylphosphoramidothioate |
| buthidazole | 3-[5-(1,1-dimethylethyl)-1,3,4-thiadiazol-2-yl]-4-hydroxy- |
| | 1-methyl-2-imidazolidinone |
| butralin | 4-(1,1-dimethylethyl)-N-(1-methylpropyl)-2,6-dinitrobenzenamine |
| buturon | N′-(4-chlorophenyl)-N-methyl-N-(1-methyl-2-propynyl)urea |
| butylate | S-ethyl bis(2-methylpropyl)carbamothioate |
| cacodylic acid | Dimethyl arsinic acid |
| cambendichlor | (phenylimino)di-2,1-ethanediyl bis(3,6-dichloro-2-methoxybenzoate) |
| carbetamide | N-ethyl-2-[[(phenylamino)carbonyl]oxy]propanamide (R)-isomer |
| CDAA | 2-chloro-NN-di-2-propenylacetamide |
| carfentrazone | “the alpha character”2-dichloro-5-[4-(difluoromethyl)-4,5-dihydro-3- |
| | methyl-5-oxo-1H-1,2,4-triazol-l-yl]-4-fluorobenzenepropanoic acid |
| CDEA | 2-chloro-N,N-diethylacetamide |
| CDEC | 2-chloro-2-propenyl diethylcarbamodithioate |
| CEPC | 2-chloroethyl (3-chlorophenyl)carbamate |
| chloramben | 3-amino-2,5-dichlorobenzoic acid |
| chlorazine | 6-chloro-N,N,N′,N′-tetraethyl-1,3,5-triazine-2,4-diamme |
| chlorbromuron | N′-(4-bromo-3-chlorophenyl)-N-methoxy-N-methylurea |
| chlorbufam | 1-methyl-2-propynyl (3-chlorophenyl)carbamate |
| chlorflurenol | 2-chloro-9-hydroxy-9H-fluorene-9-carboxylic acid |
| chlonmuron | 2-[[[[(4-chloro-6-methoxy-2- |
| | pyrimidinyl)amino]carbonyl]arnino]sulfonyl]benzoic acid |
| chloroxuron | N′-[4-(4-chlorophenoxy)phenyl]-N,N-dimethylurea |
| chlorpropham | 1-methylethyl3-chlorophenylcarbamate |
| chlorsulfuron | 2-chi oro-N-[[(4-methoxy-6-methyl- 1,3,5-tnazin-2-yl) |
| chlorthiamid | 2,6-dichlorobenzenecarbothiamide |
| chlortoluron | N′-(3-chloro-4-methylphenyl)-N,N-dimethylurea |
| cinmethylin | exo-(f)-1-methyl-4-(1-methylethyl)-2-[(2-methylphenyl)methoxy]7- |
| | oxabicyclo[2.2.1]heptane |
| cisanilide | cis-2,5-dimethyl-N-phenyl-1-pyrrolidinecarboxamide |
| clethodim | (E,E)-(f)-2-[1-[[(3-chloro-2-propenyl)oxy]imino]propyl]-5-[2- |
| | (ethylthio)propyl]-3-hydroxy-2-cyclohexen-1-one |
| clofop | 2-[4-(4-chlorophenoxy)phenoxy]propanoic acid |
| clomazone | 2-[(2-chlorophenyl)methyl]-4,4-dimethyl-3-isoxazolidinone |
| cloproxydim | (E,E)-2-[1-[[(3-chloro-2-propenyl)oxy]amino]butyl]-5-[2- |
| | (ethylthio)propyl]-3-hydroxy-2-cyclohexen-1-one |
| cloransulam | 3-chloro-2-[[(5-ethoxy-7-fluoro[1,2,4]triazolo[1,5-c]pyrimidin- |
| | 2yl)sulfonyl]amino]benzoic acid |
| clopyralid | 3,6-dichloro-2-pyridinecarboxylic acid |
| CMA | calcium salt of MAA |
| copper sulfate | copper sulfate |
| 4-CPA | (4-chlorophenoxy)acetic acid |
| 4-CPB | 4-(4-chlorophenoxy)butyric acid |
| CPMF | 1-chloro-N′-(3,4-dichlorophenyl)-N-N-dimethylformarnidine |
| 4-CPP | 2-(4-chlorophenoxy)propionic acid |
| CPPC | 2-chloro-l-methylethyl (3-chlorophenyl)carbamate |
| cyanazine | 2-[[4-chloro-6-(ethylamino)-1,3,5-triazin-2-yl]amino]-2- |
| | methylpropanenitrile |
| cycloate | S-ethyl cyclohexylethylcarbamothioate |
| cyclosulfamuron | N-[[[2-(cyclopropylcarbonyl)phenyl]amino]sulfonyl]-N′-(4,6-dimethoxy-2- |
| | pyrimidinyl)urea |
| cycluron | N′-cyclooctyl-N,N-dimethylurea |
| cyhalofop | (R)-2-[4-(4-cyano-2-fluorophenoxy)phenoxy]propanoic acid |
| cyperquat | 1-methyl-4-phenylpyridimum |
| cyprazine | 6-chloro-N-cyclopropyl-N′-(1-methylethyl)-1,3,5-triazine-2,4-diamine |
| cyprazole | N-[5-(2-chloro-1,1-dimethylethyl)-1,3,4-thiadiazol-2- |
| | yi]cyclopropanecarboxamide |
| cypromid | N-(3,4-dichlorophenyl)cyclopropanecarboxamide |
| 2,4-D | (2,4-dichlorophenoxy)acetic acid |
| 3,4-DA | (3,4-dichlorophenoxy)acetic acid |
| dalapon | 2,2-dichloropropanoic acid |
| dazomet | tetrahydro-3,5-dimethyl-2H-1,3,5-thiadiazine-2-thione |
| 2,4-DB | 4-(2,4-dichlorophenoxy)butanoic acid |
| 3,4-DB | 4-(3,4-dichlorophenoxy)butanoic acid |
| DCB | 1,2-dichlorobenzene |
| DCPA | dimethyl 2,3,5,6-tetrachloro-1,4-benzenedicarboxyl ate |
| DCU | N,N′-bis(2,2,2-drichloro-l-hydroxyethyl)urea |
| 2,4-DEB | 2-(2,4-dichlorophenoxy)ethyl benzoate |
| delachlor | 2-chloro-N-(2,6-dimethylphenyl)-N-[(2-methylpropoxy)methyl]acetamide |
| 2,4-DEP | tris[2-(2,4-dichlorophenoxy)ethyl]phosphite |
| desmedipham | ethyl [3-[[(phenylamino)carbonyl]oxy]phenyl]carbamate |
| desmetryn | N-methyl-N′-(1-methylethyl)-6-(methylthio)-1,3,5-tiiazine-2,4-diamine |
| diallate | S-(2,3-dichloro-2-propenyl) bis(1-methylethyl)carbamothioate |
| dicamba | 3,6-dichloro-2-methoxybenzoic acid |
| dichlobenil | 2,6-dichlorobenzonitrile |
| dichlormate | 3,4-dichloro benzenemethanol methylcarbamate |
| dichlorprop | (f)-2-(2,4-dichlorophenoxy)propanoic acid |
| diclofop | (f)-2-[4-(2,4-dichlorophenoxy)phenoxy]propanoic acid |
| dicryl | N-(3,4-dichlorophenyl)-2-methyl-2-propenamide |
| diethatyl | N-(chloroacetyl)-N-(2,6-diethylphenyl)glycine |
| diclosulam | N-(2,6-dichlorophenyl)-5-ethoxy-7-fluoro[1,2,4]triazolo[1,5-c]pyrimidine- |
| | 2-sulfonamide |
| difenopenten | (E)-(±)-4-[4-[4-(trifluoromethyl)phenoxy]phenoxy]-2-pentenoic acid |
| difenoxuron | N′-[-(4-methoxyphenoxy)phenyl]-N,N-dimethylurea |
| difenzoquat | 1,2-dimethyl-3,5-diphenyl-1H-pyrazolium |
| dimethachlor | 2-chloro-N-(2,6-dimethylphenyl)-N-(2-methoxyethyl)acetamide |
| dimethametryn | N-(1,2-dimethylpropyl)-N′-ethyl-6-(methylthio)1,3,5-tf azine-2,4-diamine |
| dinitramine | N3,N3-diethyl-2,4-dinitro-6-(trifluoromethyl)-1,3-benzenediamine |
| dinosam | 2-(1-methylbutyl)-4,6-dinitrophenol |
| dinoseb | 2-(1-methylpropyl)-4,6-dimtrophenol |
| dinoterb | 2-(1,1-dimethylethyl)-4,6-dimtrophenol |
| diphenamid | N,N-dim ethyl -a-phenyl benzeneacetamide |
| dipropetryn | 6-(ethylthio)-N,N′-bis(1-methyl ethyl)-1,3,5-tnazine-2,4-diamine |
| diquat | 6,7-dihydrodipyrido[1,2-a: 2′,1′-c]pyrazinediium ion |
| dithiopyr | S,S-dimethyl 2-(difluoromethyl)-4-(2-methylpropyl)-6-(trifluoromethyl)- |
| | 3,5-pyridinedicarbothioate |
| diuron | N′-(3,4-dichlorophenyl)-N,N-dimethylurea |
| DNOC | 2-methyl-4,6-dinitrophenol |
| 3,4-DP | 2-(3,4-dichlorophenoxy) propanoic acid |
| DSMA | Disodium salt of MAA |
| EBEP | Ethyl bis (2-ethylhexyl)phosphinate |
| eglinazine | N-(4-chloro-6-ethyl amino- 1,3,5-triazin-2-yl)glycine |
| endothall | 7-oxabicyclo[2. 2.1]heptane-2,3-dicarboxylic acid |
| endothal-sodium | Sodium salt of endothal |
| EPTC | S-ethyl dipropyl carbamothioate |
| erbon | 2-(2,4,5-trichlorophenoxy)ethyl-2,2-dichloropropanoate |
| ethalfluralin | N-ethyl-N-(2-methyl-2-propenyl)-2,6-dinitro-4- |
| | (trifluoromethyl)Benzenamine |
| ethametsulfuron | 2-[[[[[4-ethoxy-6-(methyl amino)- 1,3,5-tnazin-2- |
| | yl]amino]carbonyl]amino]sulfonyl]benzoic acid |
| ethidimuron | N-(5-ethylsulfonyl-1,3,4-thiadiazol-2-yl)-N,N′-dimethylurea |
| ethiolate | S-ethyl diethylcarbamothioate |
| ethofumesate | (f)-2-ethoxy-2,3-dihydro-3,3-dimethyl-5-benzofuranylmethanesulfonate |
| EXD | diethyl thioperoxydicarbonate |
| fenac | 2,3,6-trichlorobenzeneacetic acid |
| fenoxaprop | (±-)-2-[4-[(6-chloro-2-benzoxazolyl)oxy]phenoxy)propanoic acid |
| fenuron | NN-dim ethyl -N′-pbenylurea |
| fenuron TCA | salt of fenuron and TCA |
| flamprop | N-benzoyl-N-(3-chloro-4-fluorophenyl)-DL-alanine |
| fluazifop | (f)-2-[4-[[5-(trifluoromethyl)-2-pyndinyl]oxy]phenoxy]propanoic acid |
| fluazifop-P | (R)-2-[4-[[5-(trifluoromethyl)-2-pyridinyl]oxy]phenoxy]propanoic acid |
| fluchloralin | N-(2-chloroethyl)-2,6-dinitro-N-propyl-4-(trifluoromethyl)benzenamine |
| flumetsulam | N-(2,6-difluorophenyl)-5-methyl[1,2,4]triazolo[1,5-a]pynmidine-2- |
| | sulfonamide |
| flumiclorac | [2-chloro-4-fluoro-5(1,3,4,5,6,7-hexahydro-1,3-dioxo-2H-isoindol-2- |
| | yl)phenoxy]acetic acid |
| flumioxazin | 2-[7-fluoro-3,4-dihydro-3-oxo-4-(2-propynyl)-2H-1,4-benzoxazin-6-yl]- |
| | 4,5,6,7-tetrahydro-1 H-isoindole-1,3(2H)-dione |
| fluometuron | N,N-dimethyl-N′-[3-(tnfluoromethyl)phenyl]urea |
| fluorochloridone | 3-chloro-4-(chloromethyl)-1-[3-(trifluoromethyl)phenyl]2-pyrrolidinone |
| fluorodifen | 2-vitro-1-(4-nitrophenoxy)-4-trifluoromethylbenzene |
| fluoroglycofen | carboxymethyl5-[2-chloro-4-(trifluoromethyl)phenoxy]-2-mtrobenzoate |
| flupropacil | 1-methylethyl 2-chloro-5-[3,6-dihydro-3-methyl-2,6-dioxo- |
| | 4(trifluoromethyl)-1(2H)-pyrimidinyl]benzoate |
| flupyrsulfuron | 2-[[[[(4,6-dimethoxy-2-pyrimidinyl)amino]carbonyl]amino]sulfonyl)-6- |
| | (trifluoromethyl)-3 -pyridinecarboxylic acid |
| fluridone | 1-methyl-3-phenyl-5-[3-(trifluoromethyl)phenyl]-4(1 H)-pyridinone |
| fluroxypyr | [(4-amino-3,5-dichloro-6-fluoro-2-pyridinyl)oxy]acetic acid |
| flurtamone | (f)5(methylamino)2-phenyl-4-[-(trifluoromethyl)phenyl]-3(2H)furanone |
| fomesafen | 5-[2-chloro-4-(trifluoromethyl)phenoxy]-N-(methylsulfonyl)2- |
| | nitrobenzamide |
| fosamine | ethyl hydrogen (aminocarbonyl)phosphonate |
| glufosinate | 2-amino-4-(hydroxymethylphosphinyl)butanoic acid |
| glyphosate | N-(phosphonomethyl)glycine |
| halosafen | 5-[2-chloro-6-fluoro-4-(trifluoromethyl)phenoxy]N-(ethylsulfonyl)-2- |
| | mtrobenzamide |
| haloxyfop | (±)-2-[4- [[3-chloro-5-(trifluoromethyl)-2-pyndinyl]oxy]phenoxy]propanoic |
| | acid |
| hexaflurate | potassium hexafluoroarsenate |
| hexazinone | 3-cyclohexyl-6(dimethylamino)-1-methyl-1,3,5-tnazine-2,4(1H,3H)dione |
| imazamethabenz | (f)-2-[4,5-dihydro-4-methyl-4-(1-methylethyl)-5-oxo-1H-imidazol-2-yl]- |
| | 4(and 5)-methylbenzoic acid (3:2) |
| imazamox | 2-[4,5-dihydro-4-methyl-4-(1-methylethyl)-5-oxo-1H-imidazol-2-yl]-5- |
| | (methoxymethyl)-3-pyridinecarboxylic acid |
| imazapyr | (f)-2-[4,5-dihydro-4-methyl-4-(1-methylethyl)-5-oxo-1 H-imidazol-2-yl-3- |
| | pyridinecarboxylic acid |
| imazaquin | 2-[4,5-dihydro-4-methyl-4-(1-methylethyl)-5-oxo-1H-imidazol-2-yl]- |
| | 3quinolinecarboxylic acid |
| imazethapyr | 2-[4,5-dihydro-4-methyl-4-(1-methylethyl)-5-oxo-]H-imidazol-2-yl]5-ethyl- |
| | 3-pyridinecarboxyiic acid |
| ioxyml | 4-hydroxy-3,5-diiodobenzonitnle |
| ipazine | 6-chloro-N,N-diethyl-N′-(1-methylethyl)-1,3,5-triazine-2,4-diamine |
| IPX | O-(1-methylethyl)carbonodithioate |
| isocarbamid | N-(2-methylpropyl)-2-oxo-l-imidazolidinecarboxamide |
| isocil | 5-bromo-6-methyl-3-(1-methylethyl)-2,4(1 H,3H)-pyrimidinedione |
| isomethiozin | 6-(1,1-dimethylethyl)-4-[(2-methylpropylidene)amino]-3-(methylthio)- |
| | 1,2,4-triazin-5-(4H)-one |
| isopropalin | 4-(1-methylethyl)-2,6-dinitro-N,N-dipropylbenzenamine |
| isoproturon | NN-dimethyl-N′-[4-(1-m ethyl ethyl)phenyl]urea |
| isouron | N′-[5-(1,1-dimethylethyl)-3-]soxazolyl]-N,N-dimethylurea |
| isoxaben | N-[3-(1-ethyl-1-methylpropyl)-5-isoxazolyl]-2,6-dimethoxybenzamide |
| karbutilate | 3-[[(dimethylamino)carbonyl]amino]phenyl(1,1-dimethylethyl)carbamate |
| KOCN | potassium cyanate |
| lactofen | (±)-2-ethoxy-I -methyl-2-oxoethyl5-[2-chloro-4-(trifluoromethyl)phenoxy]- |
| | 2-nitrobenzoate |
| lenacil | 3-cyclohexyl-6,7-dihydro-1 H-cycIopentapyrimidine-2,4(3H,5H)-dione |
| Imuron | N′-(3,4-dichlorophenyl)-N-methoxy-N-methylurea |
| MAA | methylarsonic acid |
| MAMA | monoammomum salt of MAA |
| maleic hydrazide | 1,2-dihydro-3,6-pyndazinedione |
| MCPA - | (4-chloro-2-methylphenoxy)acetic acid |
| MCPB | 4-(4-chloro-2-methylphenoxy)butanoic acid |
| mecoprop | (f)-2-(4-chloro-2-methylphenoxy)propanoic acid |
| mefluidide | N-[2,4-dimethyl-5-[[(trifluoromethyl)sulfonyl]amino]phenyl]acetamide |
| metam-sodium | Sodium salt of metham |
| metamitron | 4-amino-3-methyl-6-phenyl-1,2,4-triazin-5(4H)-one |
| methalpropalin | N-(2-methyl-2-propenyl)-2,6-dinitro-N-propyl-4- |
| | (trifluoromethyl)benzenamine |
| metham | methylcarbamodithioic acid |
| methazole | 2-(3,4-dichlorophenyl)-4-methyl-1,2,4-oxadiazolidine-3,5-dione |
| methibenzuron | N-(2-benzothiazolyl-N,N′-dimethylureaN-(3-methoxypropyl)-N′-(l- |
| | methylethyl)-6-(methylthio)methoprotryn 1,3,5-triazine-2,4-dianiine |
| methyl bromide | Bromomethane |
| metobromuron | N′-(4-bromophenyl)-N-methoxy-N-methylurea |
| metolachlor | (2-methoxy-1-methylethyl)acetamide2-chloro-N-(2-ethyl-6-methylphenyl)- |
| | N |
| metosulam | N-(2,6-dichloro-3-methylphenyl)-5,7-dimethoxy[1,2,4]triazolo[1,5- |
| | a]pyrimidine-2- sulfonamide |
| metoxuron | N′-(3-chloro-4-methoxyphenyl)-NN-dimethyl urea |
| metnbuzin | 4-amino-6-(1,1-dimethylethyl)-3-(methylthio)-1,2,4-triazin-5(4H)-one |
| metsulfuron | 2-[[[[(4-methoxy-6-methyl-1,3,5-tiiazin-2- |
| | yl)amino]carbonyl]amino]sulfonyl]benzoic acid |
| molmate | S-ethyl hexahydro-1H-azepine-1-carbothioate |
| monalide | N-(4-chlorophenyl)-2,2-dimethylpentanamide |
| monolinuron | N′-(4-chlorophenyl)-N-methoxy-N-methylurea |
| monuron | N′-(4-chlorophenyl)-N,N-dimethylulea |
| monuron TCA | salt of monuron and TCA |
| MSMA | monosodium salt of MAA |
| napropamide | N,N-diethyl-2-(1-naphtha]enyloxy)propan am]de |
| naptalam | 2-[(1-naphthalenylamino)carbonyl]benzoic acid |
| neburon | N-butyl-N′-(3,4-dichlorophenyl)-N-methylurea |
| nicosulfuron | 2-[[[[(4,6-dimethoxy-2-pyrimidinyl)amino]carbonyl]amino]sulfonyl]N,N- |
| | dimethyl-3-pyridinecarboxamide |
| nitralin | 4-(methylsulfonyl)-2,6-dinitro-N,N-dipropylbenzenamine |
| nitrofen | 2,4-dichloro-1-(4-nitrophenoxy)benzene |
| nitrofluorfen | 2-chloro-1-(4-nitrophenoxy)-4-(trifluoromethyl)benzene |
| norea | N,N-dimethyl-N′-(octahydro-4,7-methano-1 H-inden-5-yl)urea3aa, |
| | 4a,5a,7a; 7aa-isomer |
| norflurazon | 4-chloro-5-(methylamino)-2-(3-(trifluoromethyl)phenyl)- |
| | 3(2H)pyridazinone |
| OCH | 2,3,4,4,5,5,6,6-octachloro-2-cyclohexen-1-one |
| oryzalin | 4-(dipropylamino)-3,5-dinitrobenzenesulfonamide |
| oxadiazon | 3-[2,4-dichloro-5-(l-methylethoxy)phenyl]-5-(l,l-dimethylethyl)1,3,4- |
| | oxadiazol-2-(3H)-one |
| oxyfluorfen | 2-chloro-1-(3-ethoxy-4-nitrophenoxy)-4-(trifluoromethyl)benzene |
| paraquat | 1,1′-dimethyl-4,4′-bipyridinium ion |
| PBA | chlorinated benzoic acid |
| PCP | pentachlorophenol |
| pebulate | S-propyl butylethylcarbamothioate |
| pelargonic acid | nonanoic acid |
| pendimethalin | N-(1-ethylpropyl)-3,4-dimethyl-2,6-dimtrobenzenamine |
| perfluidone | 1,1,1-trifluoro-N[2-methyl-4-(phenylsulfonyl)phenyl]methanesulfonamide |
| phemsopham | 3-[[(1-methylethoxy)carbonyl]amino]phenyl ethylphenylcarbamate |
| phenmedipham | 3-[(methoxycarbonyl)amino]phenyl (3-methylphenyl)carbamate |
| picloram | 4-amino-3,5,6-trichloro-2-pyridinecarboxylic acid |
| piperophos | S-[2-(2-methyl-l-piperidinyl)-2-oxoethyl]O,O-dipropylphosporodithioate |
| PMA | (acetato-O)phenylmercury |
| potassium azide | potassium azide |
| pnmisulfuron | 2-[[[[[4,6-bis(difluoromethoxy)-2- |
| | pyrimidinyl]amino]carbonyl]amino]sulfonyl]benzoic acid |
| procyazine | 2-[[4-chloro-6-(cyclopropylamino)-1,3,5-triazine-2-yl]amino]2- |
| | methylpropanenitrile |
| prodiamine | 2,4 dinitro-N3,N3-dipropyl-6-(trifluoromethyl)-1,3-benzenediamine |
| profluralin | N-(cyclopropyhnethyl)-2,6-dinitro-N-Propyl-4- |
| | (tnfluoromethyl)benzenamine |
| proglinazine | N-[4-chloro-6-(1-methylethylamino)-1,3,5-triazine-2-yl]glycine |
| prometon | 6-methoxy-N,N′-bis(1-methylethyl)-1,3,5-triazine-2,4-diarnme |
| prometryn | N,N′-bis(1-methylethyl)-6-(methylthio)-1,3,5-triazine-2,4-diamine |
| pronamide | 3,5-dichloro (N-1,1-dimethyl-2-propynyl)benzamide |
| propachlor | 2-chloro-N-(1-methylethyl)-N-phenylacetamide |
| propanil | N-(3,4-dichlorophenyl)propanamide |
| propaquizafop | (R)-2-[[(1-methylethylidene)amino]oxy]ethyl2-[4-[(6-chloro-2- |
| | qumoxalinyl)oxy]phenoxy]propanoate |
| propazine | 6-chloro-N,N′-bis(1-methyl ethyl)-1,3,5-triazine-2,4-diamine |
| propham | 1-methylethyl phenylcarbamate |
| prosulfalin | N-[[4-(dipropylamino)-3,5-dinitrophenyl]sulfonyl]S,S-dimethylsulfilimine |
| proxan-sodium | sodium salt of IPX |
| prynachlor | 2-chloro-N-(1-methyl-2-propynyl)-N-phenylacetamide |
| pyrazon | 5-amino-4-chloro-2-phenyl-3(2H)-pyridazinone |
| pyriclor | 2,3,5-dichloro-4-pyridinol |
| pyridate | O-(6-chloro-3-phenyl-4-pyridazinyl) S-octyl carbonothioate |
| pynthiobac | 2-chloro-6-[(4,6-dimethoxy-2-pyrimidinyl)thio]benzoic acid |
| quinclorac | 3,7-dichloro-8-quinolinecarboxylic acid |
| quinonamid | 2,2-dichloro-N-(3-chloro-1,4-dihydro-1,4-dioxo-2-naphthalenyl)acetamide |
| quizalofop | (±-)-2-[4-[(6-chloro-2-quinoxalinyl)oxy]phenoxy]propanoic acid |
| nmsulfuron | N-[[(4,6-dimethoxy-2-pyrimidinyl)amino]carbonyl]-3(ethyl sulfonyl)-2- |
| | pyridinesulfonamide |
| secbumeton | N-ethyl-6-methoxy-N′-(1-methylpropyl)-1,3,5-triazine-2,4-diamine |
| sethoxydim | 2-[1-(ethoxyimino)butyl]-5-[2-(ethylthio)propyl]3-hydroxy-2-cyclohexen- |
| | 1-one |
| sesone | 2-(2,4-dichlorophenoxy)ethyl hydrogen sulfate |
| siduron | N-(2-methylcyclohexyl)-N-phenylurea |
| silvex | 2-(2,4,5-trichlorophenoxy)propanoic acid |
| simazine | 6-chloro-NN′-diethyl-1,3,5-triazine-2,4-diamine |
| simeton | N,N′-diethyl-6-methoxy-1,3,5-triazine-2,4-diamine |
| simetryn | N,N′-diethyl-6-(methylthio)-1,3,5-triazine-2,4-diamine |
| sodium arsenite | sodium arsenite |
| sodium azide | sodium azide |
| sodium chlorate | sodium chlorate |
| solar | N-(3-chloro-4-methylphenyl)-2-methylpentanamide |
| sulfentrazone | N-[2,4-dichloro-5-[4-(difluoromethyl)-4,5-dihydro3-methyl-5-oxo-1 H- |
| | 1,2,4-triazol-1-yl]phenyl]methanesulfonamide |
| sulfometuron | 2-[[[[(4,6-dimethyl-2-pyrimidinyl)amino]carbonyl]amino]sulfonyl]benzoic |
| | acid |
| swep | methyl(3,4-dichlorophenyl)carbamate |
| 2,4,5-T | (2,4,5-tnchlorophenoxy)acetic acid |
| 2,4,5-TB | 4-(2,4,5-trichlorophenoxy)butanoic acid |
| 2,3,6-TBA | 2,3,6-trichlorobenzoic acid |
| TCA | trichloroacetic acid |
| tebuthiuron | N-[5-(I,1-dimethyl ethyl)-1,3,4-thiadiazol-2-yl]-N,N′-dimethylurea |
| terbacil | 5-chloro-3-(1,1-dimethylethyl)-6-methyl-2,4(1 H,3H)-pyrimidinedione |
| terbuchlor | N-(butoxymethyl)-2-chloro-N-[2-(1,1-dimethylethyl)6- |
| | methylphenyl]acetamide |
| terbumeton | N-(I,I-dimethylethyl)-N′-ethyl-6-methoxy-1,3,5-triazine-2,4-diamme |
| terbuthylazine | 6-chloro-N-(I,I-dimethylethyl)-N′-ethyl-1,3,5-triazine-2,4-diamine |
| terbutol | 2,6-bis(1,I-dimethylethyl)-4-methylphenyl methylcarbamate |
| terbutryn | N-(1,1-dimethylethyl)-N′-ethyl-6-(methylthio)1,3,5-tri azide-2,4-di amine |
| tetrafluron | N,N-dimethyl-N′-[3-(1,1,2,2-tetrafluoroethoxy)phenyl]urea |
| thiazafluron | NN′-dimethyl-N-[5-(trifluoromethyl)-1,3,4-thiadiazol-2-yl]urea |
| thiazopyr | methyl-2-(difluoromethyl)-5-(4,5-dihydro-2-thiazolyl)-4(2-methylpropyl)- |
| | 6-(trifluoromethyl)-3-pyridinecarboxylate |
| thifensulfuron | 3-[[[[(4-methoxy-6-methyl-1,3,5-triazin-2- |
| | yl)amino]carbonyl]amino]sulfonyl]-2-thiophenecarboxylic acid |
| thiobencarb | S-[(4-chlorophenyl)methyl]diethylcarbamothioate |
| 2,2,3-TPA | 2,2,3-trichloropropiomc acid |
| triallate | S-(2,3,3-trichloro-2-propenyl) bis(1-methylethyl)carbamothioate |
| triasulfuron | 2-(2-chloroethoxy)-N-[[(4-methoxy-6-methyl-1,3,5-triazin-2- |
| | yl)amino]carbonyl]benzenesulfonamide |
| tnbenuron | 2-[[[[(4-methoxy-6-methyl-1,3,5-triazin- |
| | 2yl)methylamino]carbonyl]amino]sulfonyl]benzoic acid |
| tricamba | 2,3,5-trichloro-6-methoxy benzoic acid |
| triclopyr | [(3,5,6-trichloro-2-pyridinyl)oxy]acetic acid |
| tndiphane | 2-(3,5-dichlorophenyl)-2-(2,2,2-trichloroethyi)oxirane |
| trietazine | 6-chloro-N,N,N′-triethyl-1,3,5-triazine-2,4-diamine |
| trifluralin | 2,6-dinitro-N,N-dipropyl-4-(trifluoromethyl)benzenamine |
| triflusulfuron | 2-[[[[[4-(dimethylamino)-6-(2,2,2-trifluoroethoxy)-1,3,5triazin-2- |
| | yl]amino]carbonyl]amino]sulfonyl]-3-methylbenzoic acid |
| trimeturon | methyl N-(4-chlorophenyl)-N,N-dimethylcarbamidate |
| tritac | 1-[(2,3,6-trichlorophenyl)methoxy]-2-propanol |
| vernolate | S-propyl dipropylcarbamothioate |
| xylachlor | 2-chloro-N-(2,3-dimethylphenyl)-N-(1-methylethyl)acetamide |

* source http://piked2.agn.uiuc.edu/wssa/subpagesiherbicide/herbtab.htm

Also within the scope of the present invention is the stimulation of the activity of an ecto-phosphatase and an ABC transporter by the over-expression of a regulatory molecule which may act by up-regulating the expression levels or by post-translationally modifying the ecto-phosphatase and the ABC transporter. Such activating regulatory molecules (e.g. calmodulin) may be over-expressed alone or together with the over-expression of the ectoapyrase and the ABC transporter or any other combination.

Particular embodiments of the invention include polynucleotides that encode MDR-ABC transporter polypeptides, ecto-phosphatase polypeptides, and stimulatory regulatory polypeptides which are capable of stimulating the efflux of drug molecules from the cells, thus conferring drug resistance. The term polynucleotide encompasses nucleic acid molecules that encode a complete protein, as well as nucleic acid molecules that encode peptides, polypeptides, or fragments of a complete protein. The polynucleotides may comprise the wild-type allele (or a portion of such an allele) of a functional peptide ABC transporter and ecto-phosphatase, or they may comprise a mutated allele of such genes. The preferred polynucleotides encode the wildtype plant, Arabidopsis thaliana, AtPGP-1 ABC transporter (GenBank accession # X61370); wild-type Homo sapiens Pgp ABC transporter (GenBank accession # M29432); wild-type Homo sapiens 1VIRP-p ABC transporter (PCT WO 98/46736); wild-type yeast, Saccharomyces cerevisiae, transporter STS I (GenBank accession # X75916); wild-type yeast, Saccharomyces cerevisiae, transporter Pdr5p (GenBank accession # 1420383); wild-type Aspergillus fumigatus Afiu-MDR1 ABC transporter (U.S. Pat. No. 5,705,352); wild-type bacterial, Lactococcus lactis, transporter LmrA (GenBank accession # U63741); wild-type plant, Pisum sativum, ectophosphatase, apyrase (GenBank accession # Z32743); and for wild-type Homo sapiens apyrase (GenBank accession # AF034840); other ecto-phosphatases include Homo sapiens CD39L2 (GenBank accession # AF039916); Homo sapiens CD39L3 (GenBank accession # AF039917); Homo sapiens CD39L4 (GenBank accession # AF039918); and Homo sapiens ATP diphosphohydrolase (GenBank accession # HSU87967).

In one embodiment of the invention, the polynucleotides are operably linked to regulatory sequences sufficient to permit the expression of the polynucleotide in a host cell. Such polynucleotides may be incorporated into nucleic acid vectors that are sufficient to permit either the propagation or maintenance of the polynucleotide within a host cell, and expression therein. The nature of the regulatory elements will depend upon the host cell, and the desired manner of expressing the polynucleotides.

The invention particularly contemplates providing the polynucleotides to plants. Suitable plants include, but are not limited to, species from the genera Fragaria, Lotus, Medicago, Onobrychis; Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Helianthus, Lactuca, Bromus, Asparagus, Antirrhinum, Hemerocallis, Nemesia, Pelargonium, Panicum, Pennisetum, Ranunculus, Senecio, Salpiglossis, Cucumis, Bromelia, Glycine, Lolium, Zea, Triticum, Sorghum, Ipomoea, Passifora, Cyclamen, Malus, Prunus, Rosa, Rubus, Populus, Santalum, Allium, Lilium, Narcissus, Ananas, Arachis, Phaseolus, Pisum, Oryza, Hordeum, Gossium.

Preferred prokaryotic vectors for subcloning and production of DNA include plasmids such as those capable of replication in E. coli such as, for example, pBR322, ColE1, psC101, pACYC184, such as those disclosed by Maniatis et al., (1982); pET 11 a, pET3a, pETI Id, pET3d, pET22d, pET12a, pET28a, and other pET variants (Novagen); pCDNA3, pCDNAI (InVitrogen).

A variety of methods may be used to introduce the polynucleotides of the present invention into a plant cell. Some examples include, but are not limited to, microinjection directly into the plant embryo cells or introduced by electroporation as described in Fromm et al., (1985); direct precipitation using polyethylene glycol as described in Paszkowski et al., (1984); in the case of monocotyledonous plants, transformation of pollen with total DNA or an appropriate functional clone and the pollen can then be used to produce progeny by sexual reproduction; introduction of polynucleotides with the Ti plasmid of Agrobacterium tumefaciens which provides a means for introducing DNA into plant cells (Horsch et al., 1988); introduction of polynucleotides with the cauliflower mosaic virus (CaMV) (U.S. Pat. No. 4,407,956).

A particularly useful Ti plasmid-based vector is Pkylx71. Schardl et al., (1987). This vector utilizes the natural transfer properties of the Ti plasmid. A cloning vehicle such as pKYLX71 allows the insertion of a polynucleotide sequence into the expression cassette by a single recombination event.

The introduction of the transferred DNA (T-DNA) of the plasmid is accomplished by infecting root calli from Ws ecotype Arabidopsis thaliana with Agrobacterium tumefaciens under kanamycin selection. The calli are then developed further into plants (Valvekens, 1992). Alternatively, shoot explants may be infected with the Agrobacterium tumefaciens bacteria. Under appropriate conditions, a ring of calli forms around the cut surface which is then transferred to growth medium, allowed to form shoots, roots and develop further into plants (Hooykass et al., (1984). Another alternative is to produce transformed plants using free DNA delivery. All plants from which protoplasts can be isolated and cultured to give whole regenerated plants can be transformed by the present invention so that whole plants are recovered which contain the introduced polynucleotide. Methods for generating plants from cultured protoplasts are described by Binding (1985), incorporated herein by reference.

Efficient plant promoters that may be used to over-express the ABC transporters and the ecto-phosphatases include over-producing plant promoters such as the small subunit (ss) of the ribulose 1, 5 biphosphate carboxylase from soybean (Berry-Lowe et al., 1982), the promoter of the chlorophyll a/b binding protein, and the CaMV promoter.

Parts obtained from the recombinant plant such as flowers, seeds, leaves, branches, bark, fruit, etc, are covered by the invention. Progeny, variants, and mutants of the recombinant plants are also included within the scope of this invention.

Conference of Drug Resistance in Microorganisms

The present invention is also directed to a method for the conference of drug resistance to microorganisms, including yeast and bacteria in part through the manipulation of the ATP gradient across biological membranes. In yeast and bacteria, the manipulation of extracellular ATP levels and the ATP gradient across biological membranes by the overexpression of a MDR-ABC transporter and/or an ecto-phosphatase may result in resistance to certain drugs. Such resistance is useful for the growth of microorganisms for biotechnological applications, e.g., those used in heterologous protein production.

It is particularly advantageous to be able to produce microorganisms which are resistant to a variety of drugs for large scale fermentation procedures where contamination by microorganisms from the environment may threaten a costly procedure. Additionally, the present invention is useful to create resistant microorganism strains in small scale fermentation processes, industrial applications, as well as in selection systems for the production of recombinant microorganisms for research applications. Research applications may include the use of resistant microorganism strains to study alternative pathways, other than antibiotics, antifungal reagents, or other commonly used drugs which could effectively inhibit the growth of microorganisms involved in disease states of humans and animals.

In yeast, a system which could confer drug resistance may be preferred to current research techniques which utilize yeast strains deficient for certain amino acid production pathways. These deficient yeast are used to introduce foreign nucleic acids of interest having a nucleotide sequence encoding a protein or proteins capable of resurrecting a deficient amino acid production pathway. Selection occurs when the yeast is grown in media deficient in that particular amino acid. This method of conferring resistance to yeast may be costly, however, since this requires that the yeast be grown in expensive cocktails of the amino acids in which they are deficient. In certain embodiments of the present invention, a cloning system in yeast confers drug resistance to the yeast coupled to the introduction of a nucleic acid molecule of interest. Such resistance may be constitutive or inducible. The yeast may then be selected by the introduction of inexpensive drugs to which the recombinant yeast would be resistant.

In other embodiments of the invention, bacteria may be produced with increased resistance to certain drugs in order to facilitate the production and to provide a system which allows for selection of bacteria based on another mechanism other than antibiotic resistance. Such resistance may be constitutive or inducible and may be particularly useful in large scale fermentation where contamination by other microorganisms is more likely to occur. Also contemplated by the present invention is the development of microorganisms which grow in soil (soil flora), particularly those designed to interact with herbicide resistant plants. The soil flora may be engineered with the same resistance to toxins as the plants with which they are engineered to react.

Additionally, the invention is directed to the development of microorganisms which are resistant to multiple toxins (two-stage resistant microorganisms or multiple-stage resistant microorganisms). The toxins could be presented to such two-stage resistant organisms or multiple-stage microorganisms simultaneously or at independent times. The present invention also contemplates the development of two-stage or multiple-stage resistant plants.

In one embodiment of the invention, the over-expression of an ectophosphatase confers drug resistance in wild-type or genetically engineered microorganisms. This effect was seen in yeast cells over-expressing plant apyrase grown in the presence of cycloheximide, a potent inhibitor of protein expression.

In another embodiment of the invention, the over-expression of an ABC transporter confers drug resistance in wild-type and genetically engineered microorganisms. In a preferred embodiment, the ABC transporter which is over-expressed is the Arabidopsis thaliana ABC transporter AtPGP-1. This ABC transporter was able to confer resistance to yeast cells grown in the presence of cycloheximide.

In a further embodiment of the invention the affect of over-expression of both an MDR-ABC transporter and an ecto-phosphatase is to enhance the ATP gradient across biological membranes and thus stimulate the resistance to certain antimicrobial agents. In a particularly preferred embodiment of the invention the MDR-ABC transporter which is overexpressed is the Arabidopsis thaliana AtPGP-1 and the ecto-phosphatase that is over-expressed is Pisulm sativum apyrase.

The invention particularly contemplates, but is not limited to, the conference of resistance in microorganisms to cycloheximide, antibiotics, antifungal agents, pheromones, heavy metals, fluorescent dyes, DNA intercalating agents, products of plant secondary metabolism such as polyphenolics and alkaloids, plant growth substances with antimicrobial properties, and the chemicals listed in Table 1 above.

In one embodiment of the invention, the nucleic acids are operably linked to regulatory sequences sufficient to permit the transcription of the nucleic acid in the microorganism of interest. Such constructs may be incorporated into nucleic acid vectors that are sufficient to permit either the propagation or maintenance of the nucleic acid and expression thereof within the host cell. The nature of the regulatory elements is dependent upon the host cell, and the desired manner of expressing the nucleic acid (e.g. constitutively or inducibly).

The invention particularly contemplates providing the nucleic acids of interest to bacteria and yeast. Suitable bacteria include both archaebactena, which are found in incommodious environments such as bogs, ocean depths, salt brines, and hot acid springs (e.g. sulfur bacteria, extreme halophiles, methanogens), and eubacteria, which are the commonly encountered forms that inhabit soil, water, and larger living organisms (e.g. gram positive, anaerobic, blue-green algae, gram negative, and spirochetes). In a preferred embodiment, the bacteria are Escherichia coli. Suitable yeast include a large group of disparate organisms. Preferred species include the budding yeast, Saccharomyces cerevisiae, and the fission yeast, Schizosaccharomyces pombe.

Preferred prokaryotic vectors include, but are not limited to, plasmids such as those capable of replication in E. coli, for example, pBR322, ColE1, psC101, pACYC 184 such as those disclosed by Maniatis et al. (1982); pETI 1a, pET3a, pETI Id, pET3d, pET22d, pET12a, pET28a, and other pET variants (Novagen); pCDNA3, pCDNAI (InVitrogen); pRR54, pRS303, pEGFP-1, pBluescript SK, pTrc99A, B, C and their derivatives (In: Current Protocols in Molecular Biology, 1998); pGEX variants (Pharmacia) and bacteriophages (e.g. Lambda phages).

Preferred yeast vectors include plasmids such as those capable of replication in either Saccharomyces cerevisiae of Schizosaccharomyces pombe. These vectors include, but are not limited to, pYES2, pVT101, Yip5, Prp7, Yrp17, Pep13, Yep24, Ycp19, Ycp50, Ylp21, pYAC3, 2pm, pLG670 (In: Current Protocols in Molecular Biology, 1998).

A variety of methods may be used to introduce the polynucleotide sequences into a microorganism. In bacteria for example, techniques such as transformation of plasmid DNA using calcium chloride competent cells, high efficiency competent cells, electroporation, or infection by bacteriophages as described (In: Current Protocols in Molecular Biology, 1998) may be used.

In yeast, methods to introduce polynucleotides can include, but are not limited to, the introduction of polynucleotides by integrative transformation, transformation by electroporation, spheroplast transformation, transformation using lithium acetate as described (In: Current Protocols in Molecular Biology, 1998) and PEG lithium acetate transformation procedure (Eble, 1992).

Also within the scope of the present invention is the conference of drug resistance to eukaryotic cell lines grown in tissue culture, including insect cell lines and mammalian cell lines. The conference of drug resistance to eukaryotic cell lines may be useful in the use of such cell lines for the production of recombinant proteins, the study of chemotherapeutic resistance in cells from various sources, and in the study of toxic levels of drugs in certain resistant cell lines.

Preferred eukaryotic vectors include but are not limited to, viral vectors, naked nucleic acids, plasmids, shuttle vectors, complexes of nucleic acids and other molecules, such as polycations (e.g. cationic lipids), including those described (In: Current Protocols in MolecuIar Biology, 1998) for introduction of heterologous DNA in mammalian cells and those described in Baculovirus Expression Vectors, (1994) for introduction of heterologous DNA in insect cells.

Inhibition of Drug Resistance in Chemotherapy

In an alternative embodiment of the invention, modulation of the ATP gradient, specifically the suppression of the gradient, may be achieved by inhibiting the activity of the ecto-phosphatase and the ABC transporter. Suitable inhibitor mechanisms include, but are not limited to, the use of small molecules which may bind to and inhibit the activity of the ectophosphatase and small molecules which may bind to and inhibit the ABC transporter. Other suitable inhibitor mechanisms include, but are not limited to, the expression of anti-sense RNA molecules which may inhibit the transcription or translation of ecto-phosphatases and ABC transporters, as well as the expression of dominant negative mutants of the ecto-phosphatase and the ABC transporters which may act to interfere with and inhibit the activity of their wild-type counterparts. Also within the scope of the invention is the over-expression of regulatory molecules which inhibit the activity of the ecto-phosphatase and the ABC transporter. The ectophosphatase may be inhibited alone or together with the ABC transporter.

Accordingly, it is one object of the present invention to provide molecules which down-regulate the activity of ecto-phosphatases either alone or together with ABC transporters.

The present invention provides for methods for the transcription of exogenous antisense RNA, in vivo or in vitro, comprising the administration of a polycistronic vector which may contain nucleic acid molecules from which may be transcribed an antisense RNA complementary to an ecto-phosphatase RNA molecule and an ABC transporter RNA molecule. The ecto-phosphatase nucleic acids and the ABC transporter nucleic acids may be operatively linked to a constitutive promoter or an inducible promoter (e.g. the constitutive major intermediate early promoter of cytomegalovirus or the inducible metallothionine promoter). Also within the scope of the invention are multiple vectors whereby the ecto-phosphatase nucleic acid and the ABC transporter nucleic acid are incorporated into separate vectors.

The present invention also provides for methods for the expression of exogenous regulatory molecules or small molecules, in vivo or in vitro, comprising the administration of a polycistronic vector or multiple vectors which may incorporate nucleic acid molecules encoding for regulatory proteins, or small molecules capable of inhibiting the activity of an ecto-phosphatase and an ABC transporter. These nucleic acids which encode for regulatory molecules may be operatively linked to either a constitutive promoter or an inducible promoter as described above.

Additionally, it is an object of the present invention to provide inhibitors of ecto-phosphatases (e.g. apyrase) in physiological compositions for modulating MDR states. Such physiological compositions comprise a small molecule capable of inhibiting an ecto-phosphatase and a physiologically acceptable carrier or diluent. As used herein, the term “physiologically acceptable carrier or diluent” means any and all solvents, dispersion media, antibacterial and antifungal agents, microcapsules, liposomes, cationic lipid carriers, isotonic and absorption delaying agents and the like which are not incompatible with the ecto-phosphatase inhibitors. The use of such media and agents for physiologically active substances is well known in the art. Supplementary active ingredients may also be incorporated into the compositions.

In certain preferred embodiments, only the endogenous ecto-phosphatase is inhibited by antisense RNA, regulatory proteins, or small molecules. In other embodiments of the present invention, both the endogenous ecto-phosphatase and the ABC transporter are targeted for inhibition.

In particularly preferred embodiments, the ecto-phosphatase is human apyrase (e.g. GenBank accession # AF034840, AF039916, AF039917, Al⁷039918, HSU87967) and the MDR-ABC transporter is human MDR-1, (e.g. human P-glycoprotein MDR-1, GenBank accession A M29432 and human MRP-P PCT, publication WO 98/46736).

The nucleic acid molecules from which will be transcribed antisense RNA molecules described above or the nucleic acid molecules encoding for regulatory molecules as described above may be incorporated into any suitable cloning or expression vector, operably linked to appropriate control elements (e.g. promoter elements, enhancer elements, ribosomal binding sites, polyadenylation sites, termination sites, etc.). Examples of such vectors include, but are not limited to, herpes simplex viral based vectors such as pHSV 1 (Geller et al., 1990); retroviral vectors such as MFG (Jaffee et al., 1993), and in particular Moloney retroviral vectors such as LN, LNSX, LNCX, LXSN (Miller and Rosman, 1989); vaccinia viral vectors such as MVA (Suffer and Moss, 1992); adenovirus vectors such as pJM17 (Ali et al., 1994; Berker, 1988; Wand and Finer, 1996); adeno-associated virus vectors such as AAV/neo (Mura-Cacho et al., 1992); lentivirus vectors (Zufferey et al., 1997). Such vectors may be targeted to the tumor cells of interest as described in U.S. Pat. Nos. 5,834,256, 5,843,742, 5,830,727, 5,814,500.

The inhibition of ecto-phosphatases either alone or together with the inhibition of ABC transporters is useful in the reduction of drug resistance in cells. In one embodiment of the invention, the inhibition of ecto-phosphatases either alone or together with ABC transporters results in a loss of resistance to drug molecules used in chemotherapy. In another embodiment of the invention, administration of such inhibitory molecules is in conjunction with the administration of chemotherapeutic agents in tumor cells.

Administration of the foregoing agents may be local or systemic, using a suitable physiological carrier. Other compounds which aid in the uptake or stability of these agents, or which have beneficial activity, may also be included in the formulations of the invention. Certain embodiments of the invention thus provide pharmaceutical composition comprising an ecto-phosphatase inhibitor. By contacting a tumor cell either alone with the ecto-phosphatase inhibitor or in combination with one or more chemotherapeutic agents, therapeutic benefit may be obtained. Particular benefit may be obtained where a tumor cell is resistant to at least one chemotherapeutic agent. This may enhance the overall anti-tumor activity achieved by therapy, and/or may be used to prevent or combat multi-drug tumor resistance.

A variety of chemotherapeutic agents are suitable for use with the invention and are known to those of skill in the art. As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Further useful agents include, for example, compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors also may be used. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU is applicable in a wide range of carriers, including topical, with intravenous administration in doses ranging from 3 to 15 mg/kg/day being commonly used.

Exemplary and non-limiting chemotherapeutic agents that may be used in accordance with the invention are listed in Table 2. Accordingly, compositions comprising these agents and one or more ecto-phosphatase inhibitor(s) provided by the invention form one part of the invention, as do methods for the administration thereof. Each of the agents listed below are exemplary and by no means limiting. In this regard, the skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

TABLE 2
Chemotherapeutic Agents Useful In Neoplastic Disease

| | | NONPROPRIETARY | |
|---|---|---|---|
| | | NAMES | |
| CLASS | TYPE OF AGENT | (OTHER NAMES) | DISEASE |
| Alkylating Agents | Nitrogen Mustards | Mechlorethamine (HN₂) | Hodgkin's disease, non-Hodgkin's |
| | | | lymphomas |
| | | Cyclophosphamide | Acute and chronic lymphocytic |
| | | Ifosfamide | leukemias, Hodgkin's disease, |
| | | | non-Hodgkin's lymphomas, multiple |
| | | | myeloma, neuroblastoma, breast, |
| | | | ovary, lung, Wilms' tumor, cervix, |
| | | | testis, soft-tissue sarcomas |
| | | Melphalan (L-sarcolysin) | Multiple myeloma, breast, ovary |
| | | Chlorambucil | Chronic lymphocytic leukemia, |
| | | | primary macroglobulinemia, |
| | | | Hodgkin's disease, non-Hodgkin's |
| | | | lymphomas |
| | Ethylenimenes and | Hexamethylmelamine | Ovary |
| | Methylmelamines | Thiotepa | Bladder, breast, ovary |
| | Alkyl Sulfonates | Busulfan | Chronic granulocytic leukemia |
| | Nitrosoureas | Carmustine (BCNU) | Hodgkin's disease, non-Hodgkin's |
| | | | lymphomas, primary brain tumors, |
| | | | multiple myeloma, malignant |
| | | | melanoma |
| | | Lomustine (CCNU) | Hodgkin's disease, non-Hodgkin's |
| | | | lymphomas, primary brain tumors, |
| | | | small-cell lung |
| | | Semustine | Primary brain tumors, stomach, colon |
| | | (methyl-CCNU) | |
| | | Streptozocin | Malignant pancreatic insulinoma, |
| | | (streptozotocin) | malignant carcinoid |
| | Triazines | Dacarbazine (DTIC; | Malignant melanoma, Hodgkin's |
| | | dimethyltriazenoimidaz | disease, soft-tissue sarcomas |
| | | olecarboxamide) | |
| Antimetabolites | Folic Acid Analogs | Methotrexate | Acute lymphocytic leukemia, |
| | | (amethopterin) | choriocarcinoma, mycosis fungoides, |
| | | | breast, head and neck, lung, |
| | | | osteogenic sarcoma |
| | Pyrimidine Analogs | Fluouracil (5-fluorouracil; | Breast, colon, stomach, pancreas, |
| | | 5-FU) | ovary, head and neck, urinary |
| | | Floxuridine | bladder, prernalignant skin lesions |
| | | (fluorode-oxyuridine; | (topical) |
| | | FUdR) | |
| | | Cytarabine (cytosine | Acute granulocytic and acute |
| | | arabinoside) | lymphocytic leukemias |
| | Purine Analogs and | Mercaptopurine | Acute lymphocytic, acute |
| | Related Inhibitors | (6-mercaptopurine; | granulocytic and chronic granulocytic |
| | | 6-MP) | leukemias |
| | | Thioguanine | Acute granulocytic, acute |
| | | (6-thioguanine; TG) | lymphocytic and chronic granulocytic |
| | | | leukemias |
| | | Pentostatin | Hairy cell leukemia, mycosis |
| | | (2-deoxycoformycin) | fungoides, chronic lymphocytic |
| | | | leukemia |
| Natural Products | Vinca Alkaloids | Vinblastine (VLB) | Hodgkin's disease, non-Hodgkin's |
| | | | lymphomas, breast, testis |
| | | Vincristine | Acute lymphocytic leukemia, |
| | | | neuroblastoma, Wilms' tumor, |
| | | | rhabdomyosarcoma, Hodgkin's |
| | | | disease, non-Hodgkin's lymphomas, |
| | | | small-cell lung |
| | Epipodophyllotoxins | Etoposide | Testis, small-cell lung and other lung, |
| | | Tertiposide | breast, Hodgkin's disease, |
| | | | non-Hodgkin's lymphomas, acute |
| | | | granulocytic leukemia, Kaposi's |
| | | | sarcoma |
| | Antibiotics | Dactinomycin | Choriocarcinoma, Wilms' tumor, |
| | | (actinomycin D) | rhabdomyosarcoma, testis, Kaposi's |
| | | | sarcoma |
| | | Daunorubicin | Acute granulocytic and acute |
| | | (daunomycin; | lymphocytic leukemias |
| | | rubidomycin) | |
| | | Doxorubicin | Soft-tissue, osteogenic and other |
| | | | sarcomas; Hodgkin's disease, |
| | | Doxorubicin | non-Hodgkin's lymphomas, acute |
| | | | leukemias, breast, genitourinary, |
| | | | thyroid, lung, stomach, |
| | | | neuroblastoma |
| | | Bleomycin | Testis, head and neck, skin, |
| | | | esophagus, lung and genitourinary |
| | | | tract; Hodgkin's disease, |
| | | | non-Hodgkin's lymphomas |
| | | Plicamycin (mithramycin) | Testis, malignant hypercalcemia |
| | | Mitomycin (mitomycin C) | Stomach, cervix, colon, breast, |
| | | | pancreas, bladder, head and neck |
| | Enzymes | L-Asparaginase | Acute lymphocytic leukemia |
| | Biological Response | Interferon alfa | Hairy cell leukemia., Kaposi's |
| | Modifiers | | sarcoma, melanoma, carcinoid, renal |
| | | | cell, ovary, bladder, non-Hodgkin's |
| | | | lymphomas, mycosis fungoides, |
| | | | multiple myeloma, chronic |
| | | | granulocytic leukemia |
| Miscellaneous | Platinum Coordination | Cisplatin (cis-DDP) | Testis, ovary, bladder, head and |
| Agents | Complexes | Carboplatin | neck, lung, thyroid, cervix, |
| | | | endometrium, neuroblastoma, |
| | | | osteogenic sarcoma |
| | Anthracenedione | Mitoxantrone | Acute granulocytic leukemia, breast |
| | Substituted Urea | Hydroxyurea | Chronic granulocytic leukemia, |
| | | | polycythemia vera, essental |
| | | | thrombocytosis, malignant melanoma |
| | Methyl Hydrazine | Procarbazine | Hodgkin's disease |
| | Derivative | (N-methylhydrazine, | |
| | | MIH) | |
| | Adrenocortical | Mitotane (o,p′-DDD) | Adrenal cortex |
| | Suppressant | Aminoglutethimide | Breast |
| Hormones and | Adrenocorticosteroids | Prednisone (several other | Acute and chronic lymphocytic |
| Antagonists | | equivalent | leukemias, non-Hodgkin's |
| | | preparations available) | lymphomas, Hodgkin's disease, |
| | | | breast |
| | Progetins | Hydroxyprogesterone | Endometrium, breast |
| | | caproate | |
| | | Medroxyprogesterone | |
| | | acetate | |
| | | Megestrol acetate | |
| | Estrogens | Diethylstilbestrol | Breast, prostate |
| | | Ethinyl estradiol (other | |
| | | preparations available) | |
| | Antiestrogen | Tamoxifen | Breast |
| | Androgens | Testosterone propionate | Breast |
| | Androgens | Fluoxymesterone (other | |
| | | preparations available) | |
| | Antiandrogen | Flutamide | Prostate |
| | Gonadotropin-releasing | Leuprolide | Prostate |
| | hormone analog | | |

Inhibition of Drug Resistance in Microorganisms to Treat Infection

The present invention also relates to methods for inhibiting or ameliorating infection in animals and humans caused by microorganisms, particularly bacterial and fungal infections using inhibitory mechanisms against an ecto-phosphatase and an ABC transporter and modifying the ATP gradient across biological membranes. The invention is useful in the inhibition or amelioration of a wide range of infections including, but not limited to, gram negative bacterial infection including gram-negative sepsis, gram-negative endotoxin-related hypotension and shock, rabies, cholera, tetanus, lymes disease, tuberculosis, Candida albicans, Chlamydia, etc. The invention is based, in part, on the unexpected result that when mutant yeast deficient in two potent extracellular ATP phosphatases were cultured in cycloheximide, they were not able to grow. Surprisingly, they were rescued by the over-expression of a plant MDRABC transporter AtPGP-1, suggesting that the inability to grow in the drug was caused by an inability to efflux the drug which was coupled to a deficiency in extracellular ATP phosphatase activity.

Drug sensitivity in microorganisms may be achieved by introducing nucleic acid molecules into bacteria and yeast (as described above) that are capable of conferring inhibition of the activity of an endogenous ecto-phosphatase and an ABC transporter. Such nucleic acid molecules may transcribe an antisense RNA complimentary to endogenous RNA for an ecto-phosphatase or an ABC transporter, encode for inhibitory regulatory proteins, or encode for inhibitory drug molecules. The inhibition or amelioration of the infections may involve the administration of an anti-microbial agent (such as an antibiotic or an antifungal agent) with the concurrent administration of the aforementioned nucleic acid molecules (which may be achieved through bacteriophages, etc). Additionally, inhibitors of ecto-phosphatases or ABC transporters may be administered via a physiologically acceptable carrier as described above. In one embodiment of the invention, the inhibitor of ecto-phosphatase is selected from the compounds represented by formulas I-XIX.

Additionally, the present invention is useful in the development of genetic and epigenetic systems in humans for resistance to toxins from biological and non-biological sources. Such sources include, but are not restricted to, pathogens produced by microbial infections, pathogens and toxins derived from biological sources through human contrivance, environmental toxins not produced through biological action, and toxic substances created synthetically. In a particular embodiment, humans at risk for exposure would be vaccinated either with a gene therapy designed to bolster endogenous ATP gradients in human cells, or a chemical substance capable of enhancing the strength of the ATP gradient. In both instances, the target of the genetic or chemical therapy would be either the ABC transporter activity, ecto-phosphatase activity or both. In another embodiment of the invention, only the ABC transporter activity or the ecto-phosphatase activity in an infecting organism is diminished to inhibit drug efflux. Recombinant techniques may be used to introduce DNA sequences to the microorganism which encode for a small inhibitory molecule to either an ABC transporter or an ecto-phosphatase or both to cause the inhibition of drug efflux from the microorganism.

Certain aspects of the current invention thus concern the inhibition of bacterial cell growth contacting the bacterial cell with an inhibitor of an ecto-phosphatase either with or without contemporaneous administration of other agents capable of inhibiting bacterial growth or killing bacteria. In this manner, therapeutic benefit may be obtained for the treatment of bacterial infections. Examples of types of bacteria that could be inhibited and bacterial infections that could potentially be treated or prevented with the invention, include, but are not limited to, the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also find use, for example, against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like.

Ecto-phosphatase Inhibition

Since ecto-phosphatases have been shown by the present invention to be important actors in the modulation of the ATP gradient across biological membranes and thus useful in a variety of applications (e.g. the modulation of drug resistance), it is an object of the present invention to provide methods and assays for the identification of inhibitors of ecto-phosphatases (e.g. apyrase).

A high-throughput screen was developed to rapidly identify potential inhibitors for ecto-phosphatases and is described below in Example 7. This high-throughput screen is particularly useful, since no known specific inhibitors of the apyrase enzyme exist. Using the high throughput screen, ecto-phosphatase inhibitors are isolated by screening a small molecule library (e.g. a combinatorial library) for inhibitory activity to ecto-phosphatase (e.g. apyrase) activity. Once ecto-phosphatase inhibitory molecules are isolated from such a screen, the inhibitors may be further tested for their ability to specifically inhibit the ATPase activity of the ecto-phosphatase and to reduce drug resistance in cells.

The ecto-phosphatase inhibitory molecules of the present invention are chemically stable and physiologically active and include, inter alia, those molecules represented by Formulae I through XIX below.

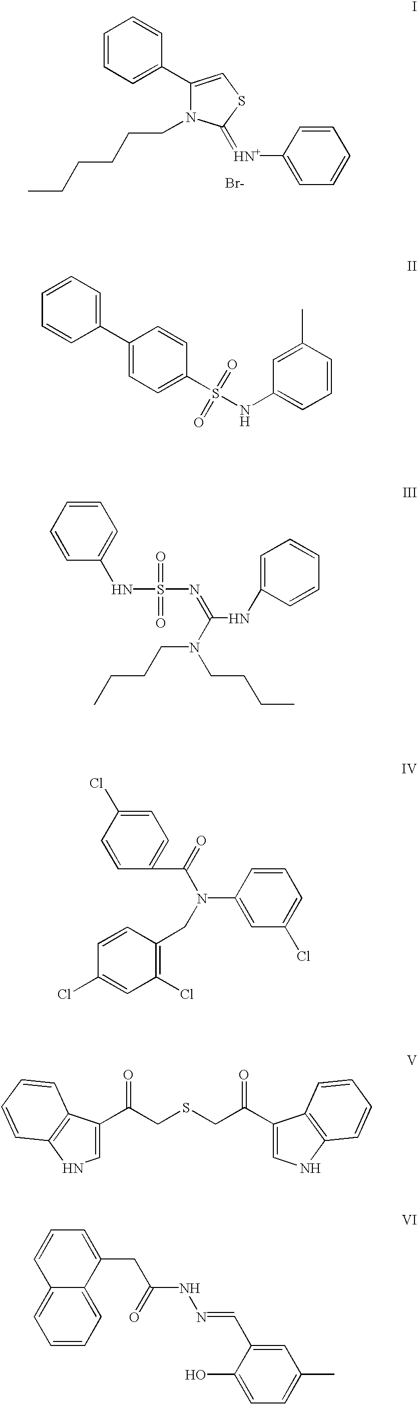

Preliminary pharmacophore studies revealed that the small molecules represented by Formulae I through XIX fall into five classes of compounds (sulfanamides, guamdines, aminothiazoles, thioketones and benzamides). Most of these chemical classes are found in other physiologically-active compounds, including those having pharmaceutical and therapeutic use. For example, sulfanamides are widely used as antibiotics. Additionally, studies for the isolation of small molecules capable of reversing MDR have described molecules belonging to two of the classes of molecules of the present invention (Medina et al., 1998; Dhamant et al., 1992). The molecules described by Medina et al. have been shown to affect MDR and the mode of action of the molecules is believed to involve tubulin interactions. The thiazine derivatives described by Dhamant et al. reverse the resistance in tumor cells to vincristine.

The ecto-phosphatase inhibitory molecules of the present invention are useful in reversing MDR in Arabidopsis plants and yeast. MDR reversal in plants and yeast cells may be shown by growing the cells in the presence of relevant drugs and in the presence and absence of the inhibitor. Cells which cannot grow in drug, in the presence of an ecto-phosphatase inhibitor, have a reversal in MDR. Additionally, the ecto-phosphatase inhibitory molecules of the present invention are useful in reversing drug resistance in mammalian cell lines (e.g. normal COS-7 cells and breast cancer tumor cells (e.g. HS5787, NM231 and MB435)) grown in the presence of a drug (e.g. a chemotherapeutic agent). MDR reversal in mammalian cells may be shown by using the fluorescent compound calcein-AM. Esterases present in cells cleave the aceto-methoxy ester (AM) from the calcein-AM and liberate calcein. Calcein is a fluorescent compound which is excitable by the 488 nm laser of a FACSCaliber flow cytometer (Becton Dickenson, Franklin Lakes, N.J.), while the uncleaved calcein-AM is not excitable. Wild type cells incubated in the presence of calcein-AM show a high level of fluorescence while MDR state cells, which efflux the calcein-AM faster than the cellular esterases can cleave it, do not show a high level of fluorescence. The mammalian cells can be tested for the reversal of MDR with the ectophosphatase inhibitors of the present invention by the amount of calcein fluorescence detected in the cells. Furthermore, the relative importance of the mammalian MDR gene and the mammalian apyrase gene in MDR can also be determined.

Specificity of the ecto-phosphatase inhibitors of the present invention may be tested with the screening assay described in Example 7 below. Inhibitors are tested for their ability to inhibit acid phosphatases, alkaline phosphatases, myosin phosphatases and the luciferase ATPase. The assays may be performed using techniques known in the art.

In one embodiment, the ecto-phosphatase is an apyrase and the ectophosphatase inhibitor is a molecule selected from among molecules represented by the Formulae I through XIX. In another embodiment, the ecto-phosphatase is apyrase and the ectophosphatase inhibitor is a molecule selected from among molecules represented by the Formulae I through V. In another embodiment, the ecto-phosphatase is apyrase and the ecto-phosphatase inhibitor is a molecule selected from among molecules represented by Formula I and Formula II.

The ecto-phosphatase inhibitors of the present invention which are acidic or basic in nature can form a wide variety of salts with various inorganic and organic bases or acids, respectively. These salts may be physiologically acceptable for in vivo administration in plants and animals, including humans. Salts of the acidic compounds of this invention are readily prepared by treating the acidic compound with an appropriate molar quantity of the chosen inorganic or organic base in an aqueous or suitable organic solvent and then evaporating the solvent to obtain the salt. Salts of the basic compounds of this invention can be obtained similarly by treatment with the desired inorganic or organic acid and subsequent solvent evaporation and isolation. The skilled artisan can produce salts of the small molecules of the present invention using techniques known in the art.

The skilled artisan readily can determine the amount of the ecto-phosphatase inhibitor that is required to inhibit the ecto-phosphatase by measuring ATPase activity in the presence and absence of varying amounts of the inhibitor. Phosphatase activity can be determined by assessing the dephosphorylation of ATP and liberation of phosphate as described below in Example 7. Additionally, parameters may be measured that are known to be associated with ecto-phosphatase activity to determine whether the molecule has ecto-phosphatase inhibitory activity. For example, ecto-phosphatase inhibitory activity may be measured in cells (e.g. plant, yeast, mammalian, tumor, etc. cell lines) by assessing the loss of resistance to drugs. Furthermore, the ecto-phosphatase inhibitory molecules of the present invention may be tested for specific inhibitory activity to ecto-phosphatases versus general phosphatases or for specific inhibitory activity for a particular ecto-phosphatase activity (e.g. apyrase).

Additionally, as stated above, the ecto-phosphatase inhibitory molecules of the present invention are useful in reversing MDR. Such a reversal has several applications including reducing resistance to chemotherapeutic agents in tumor cells and reducing resistance to antimicrobial agents in microorganisms.

Inhibition of ecto-phosphatases is useful in industrial applications as well. For example, one of the most sensitive and cost effective ways of determining the titer of microbia in soil, sludge, blood, food, and textiles is the luciferase assay which allows for the estimation of microbial biomass through the determination of precise concentrations of ATP. The sensitivity of the assay requires that “background” ATP or nonmicrobial ATP present in the system as a consequence of the source of the sample be separated from the ATP used in the microbe count. The removal of background ATP is accomplished using the ecto-phosphatase, apyrase. After removal of the background ATP with apyrase, the apyrase must be removed or inactivated. General techniques for removal could be improved and simplified with a method of inactivating the apyrase by adding a specific apyrase inhibitor of the present invention.

The present invention also provides physiologically acceptable compositions comprising an ecto-phosphatase inhibitor of the present invention and a physiologically acceptable carrier or diluent as described above. The use of such physiologically acceptable carriers or diluents are well-known in the art. Formulation of such physiological compositions can be made using known procedures, e.g. according to Remington's Pharmaceutical Sciences. Formulation of the compounds of the present invention may be stable under the conditions of manufacture and storage and must be preserved against contamination by microorganisms. Contamination can be avoided using antimicrobial (e.g. antibacterial and antifungal) agents.

The physiological forms of the compounds of the invention suitable for administration include sterile aqueous solutions or dispersions and sterile powders for the extemporaneous preparation of sterile injectable solutions or dispersions. Typical carriers include a solvent or dispersion medium containing, for example, water buffered aqueous solutions (i.e. biocompatible buffers), ethanol, polyols such as glycerol, propylene glycol, polyethylene glycol, suitable mixtures thereof, surfactants, and vegetable oils. Isotonic agents such as sugars or sodium chloride may be incorporated into the subject compositions.

The present invention is further illustrated by the following examples which in no way should be construed as being further limiting. The contents of all references cited throughout this application are hereby expressly incorporated by reference.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Inhibition of Antibiotic Resistant Bacteria With Ectophosphatase Inhibitors

### A. Summary of Protocol and Results

The purpose of this study was to evaluate various ectophosphatase inhibitor compounds for any potentiating effect or resistance reversal with methicillin resistant Staphylococcus aureus (MRSA). Two strains of Staphylococcus aureus were obtained from the American Type Culture Collection (ATCC). Those obtained were a previously characterized methicillin resistant strain (# 43300) and a previously characterized methicillin sensitive strain (# 29213) to serve as a control. Strains were received as lyophilized pellets and were rehydrated according to the ATCC Product Information Sheet using Trypticase Soy Broth.

Mueller-Hinton agar plates were prepared containing 1 of 6 inhibitor compounds investigated. The compounds were dissolved in DMSO at concentrations of 20 mg/ml. 25 μl of this stock solution was added to 25 mls of media just prior to cooling to solidification to produce plates containing 20 μg/ml of inhibitor compound. Control plates containing 25 μl DMSO only were also prepared in the same fashion.

For each of the two strains, 5 isolated colonies were picked from overnight culture plates and used to inoculate a 0.85% NaCl solution. The turbidity of each culture was adjusted using 0.85% NaCl until the turbidity of each matched a 0.5 McFarland turbidity standard. Each strain was swabbed onto control plates. The MRSA strain was swabbed also onto the inhibitor plates. Plates were allowed to dry for 5 minutes. A disk containing 5 μg of methicillin (obtained for the purpose of susceptibility testing from Bioanalyse Co., Ltd.) was placed onto the surface of all plates. Plates were incubated at 37° C.

Plates were read after 24 hours. It was noticed at this time that one plate, containing inhibitor compound NGXT1914 (Formula XV), was clear—there was no bacterial growth at all. All other plates had confluent growth except at the zone of inhibition of the methicillin disk.

Protocols for the studies were according to NCCLS guidelines (M2-A7, M100-S11) for antimicrobial susceptibility testing. Methodology for the serum bactericidal test was according to (Tentative Guideline) NCCLS M21-T, 1992. Methods for determining bactericidal activity of antimicrobial agents was according to (Tentative Guideline) NCCLS M26-T.1992. Methods for dilution antimicrobial susceptibility tests for bacteria that grow aerobically. 3rd ed. NCCLS M7-A3.1993. Protocols for evaluating dehydrated Mueller-Hinton agar were according to NCCLS M6-A, 1996. Performance standards for antimicrobial susceptibility testing were according to NCCLS M100-S8, 1998. The screening test for Oxacillin-resistant Staphylococci was according to NCCLS M7-A4. Performance standards for antimicrobial disk and dilution susceptibility tests for -bbacteria isolated from animals was according to (Approved Standard) NCCLS M31-A. Performance standards for antimicrobial susceptibility testing were according to NCCLS M100-S8.1998. Screening test for Oxacillin-resistant Staphylococci was according to NCCLS M7-A4 Susceptibility test panels.

### B. Confirmation of results

In order to ascertain whether the result with inhibitor NGXT1914 was artifactual, both MRSA and MSSA were tested on Mueller-Hinton agar plates containing varying concentrations of NGXT1914. First, plates were prepared as above with the following concentrations of inhibitor compound: 0 μg/ml (DMSO controls), 1 μg/ml, 5 μg/ml, 10 μg/ml, and 25 μg/ml. Inhibitor concentrations were prepared such that all plates contained 25 μl total DMSO. For each of the two strains, 5 isolated colonies were picked from fresh overnight culture plates and used to inoculate a 0.85% NaCl solution. The turbidity of each culture was adjusted using 0.85% NaCl until the turbidity of each matched a 0.5 McFarland turbidity standard. Both strains were each swabbed onto both control and inhibitor plates. Plates were allowed to dry for 5 minutes and were placed into the incubator. Plates were read after 24 hours (12/7/01). The results were as follows:

| Concentration | |
|---|---|
| (μg/ml NGXT1914) | Result |
| 25 μg/ml | No growth at all for either strain |
| 10 μg/ml | No growth at all for MRSA strain. Some |
| | small isolated colonies on the MSSA plate. |
| 5 μg/ml | Only a few small colonies on the MRSA |
| | plate. Confluent growth on the MSSA |
| | plate. |
| 1 μg/ml | Confluent growth on both plates. |
| Controls | Confluent growth on both plates. |

### C. Conclusions

Inhibitor NGXT 1914 inhibits growth of both resistant and sensitive Staph strains, however it is more effective against the methicillin resistant strain. This compound was tested with other cell types (mammalian cell lines, plants (Arabidopsis thaliana) yeast, Pseudomonas aeruginiosa) and has not inhibited growth of any cell line or organism at the concentrations tested. Since NGXT19I4 is anectophosphatase inhibitor, it is possible that Staph, particularly the MRSA, is dependent on a phosphatase that is an ideal substrate for NGXT1914. The results obtained indicated that NGXT1914 was more effective against MRSA versus MSSA than previously identified compounds.

### Example 2

### Inhibition of Chemotherapy-Resistant Tumor Cells With An Ecto-Phosphatase Inhibitor

In order to determine the effect of ecto-phosphatase inhibitors on tumor cells resistant to a chemotherapeutic agent, 2 breast cancer tumor cell lines were tested, a vinblastine resistant line (SW-13Vb003) and its vinblastine sensitive parent line, SW-13. A standard 4 day incubation at 37° C. and 5% CO₂ was used. IC₅₀ tests (MTT) were done using standard methodology in 96 well plate format with inhibitor concentrations ranging from 0-90 μg/ml (DMEM media). Results showed that 3 of the inhibitor compounds tested, NGXT194 (Formula VI), NGXT196 (Formula VIII), and NGXT1915 (Formula X), produced lower IC₅₀ values with the resistant cell line than with the sensitive line. For NGXT194, SW-13 was sensitive at 20 μg/ml, whereas the vinblastine resistant line SW-13Vb003 was sensitive at 10 μg/ml. For NGXT196, SW-13 was sensitive at 30 μg/ml, whereas the vinblastine resistant line SW-13Vb003 was sensitive at 10 μg/ml. For NGXT1915, SW-13 was sensitive at 50 μg/ml, whereas the vinblastine resistant line SW-13Vb003 was sensitive at 20 μg/ml.

Based on the inventors previous findings concerning the role of ecto-phosphatases in drug resistance, it seems likely that many resistant cells have upregulated phosphatases and that inhibiting a phosphatase critical to maintaining these cells could result in cell death. One hypothesis is that once these phosphatase enzymes are upregulated or are used in drug resistance their function becomes one that is vital to the cell. Since this phenomenon has been seen now in a bacterial resistance model it might be expected that the ectophosphatase inhibiting compounds could preferentially kill other types of resistant cells or act as biocides to certain cells.

### Example 3

### Over-Expression of Ecto-Phosphatase Does Not Increase the Cellular Uptake of Adenosine

### A. Materials and Methods

Transgenic Plant Construction: psNTP9 (Pisum Sativum apyrase, GenBank accession #Z32743) was subcloned as a Sall to Xbal fragment into pKYLX71 (Schardl et al, 1987, supra). This plasmid was transformed into A. tumefaciens GV3101 [pMP90] pKYLX71 (Koncz and Shell, 1986), which was used to infect root call from Ws ecotype Arabidopsis thaliana under kanamycin selection (Valvekens et al., 1992). Four individual lines, obtained from separate calli, were propagated to the third generation (T3).

Subcellular Apyrase Distribution in Pea: Etiolated pea plumules served as the tissue source for nuclei and cytoplasm isolation as described by Chen and Roux (Plant Physiol. 81:609-612 (1986)). Plasma membrane was prepared from 30 g of pea root tissue (Zhu Mei Jun and Chen Jia; 1995, Acta Botanica Sinica 37:942-949). Western analysis was performed on 15-30 pg of protein from cytoplasm, plasma membrane and nuclei using a polyclonal anti-apyrase antibody raised against the purified pea protein (Tong et al., 1993). To determine the orientation of the pea apyrase in the pea plasma membrane, outside-out vesicles were prepared and the accessibility of the enzyme was determined by selective trypsin proteolysis, or membrane shaving, followed by activity assays and western blotting.

Phosphate uptake experiments and growth assays: In all experiments the growth media did not contain sugar, and plants were grown in sterile culture at 22° C. under 150-200 pE of continuous light. Unless otherwise noted, a standard 0.8% agar medium (Becton Dickenson, Cockeysville, Md.) containing 100 μM phosphate was used for uptake assays (Somerville et al., 1982). Plants used for the phosphate uptake experiments were grown singly in 1 ml of the standard agar medium for 15 days prior to the experiment. On the day of the experiment, 10 μCi³²p was applied to the side of the culture dish and allowed to diff-use through the agar. The lids of 95 mm×15 mm tissue culture dishes (Fisher, Pittsburgh, Pa.) were removed to facilitate transpiration.

After 18 hours, the plants were removed from the medium. The aerial portions of the plant not in contact with the agar were weighed and counted by liquid scintillation. For each plant the entire root system was carefully pulled from the agar and washed in ice cold water prior to scintillation counting. To measure the transport of the products of ATP hydrolysis by the transgenic plants overexpressing apyrase and by wild-type plants, [2,8³H]ATP, [α³²P]ATP, and [γ³²P]ATP (Amersham) were fed to 15-day-old plants in separate treatments. All treatments were analyzed for significance in a T-test (n>4-6 for all groups, *P<0.05, error bars=s.e.m.).

### B. Results

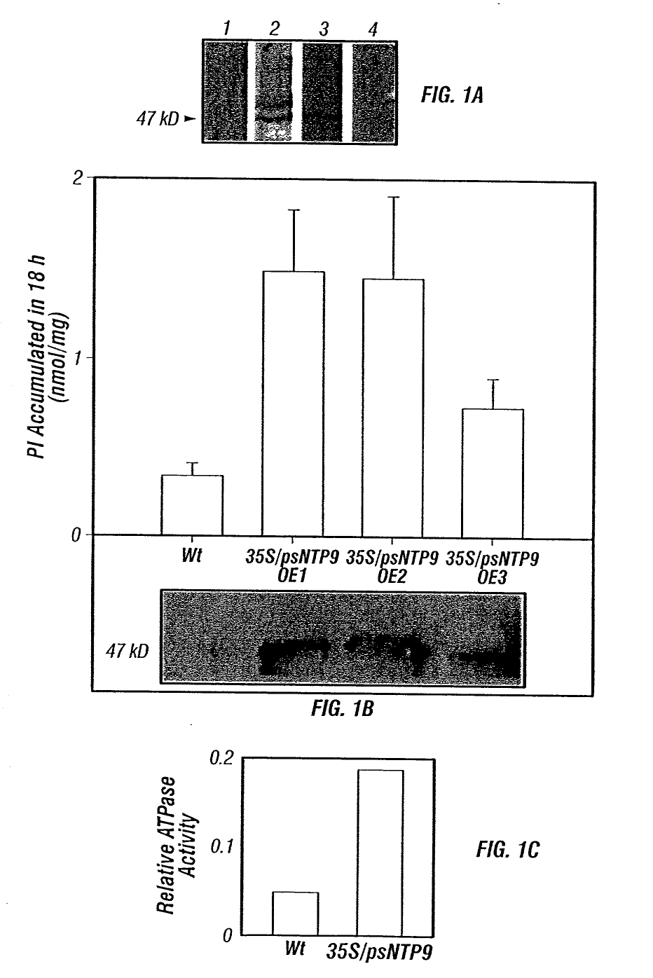

Detection of the pea apyrase in nuclei and in purified plasma membrane: By immunoblot assay, the pea apyrase was found to be associated with nuclei and with purified plasma membranes but not with the cytoplasm (FIG. 1A). The contents of the lanes in FIG. 1A are as follows: Lane 1, cytoplasm; Lane 2, purified plasma membrane; Lane 3, purified nuclei; and Lane 4, pre-immune control of nuclei. Protease treatment destroyed both apyrase activity and antigenicity in outside-out plasma membrane vesicles. After trypsin treatment, the exterior face of the vesicle showed 30% of the ecto-phosphatase activity of the untreated sample. Endo-phosphatase activities were retained after trypsin treatment, indicating that the digest occurred exclusively on the exterior face of the membrane. These data indicated that the ecto-apyrase was in fact being expressed in the extracellular matrix (ECM).

Enhanced Growth of Plants Over-Expressing Apyrase: Three of the four transgenic plant lines constitutively expressed psNTP9 under the control of the cauliflower mosaic virus 35S promoter and over an 18 hour period showed two to five times as much phosphate accumulation in shoots as wild type (FIG. 1B); Top, the total phosphate accumulated in the shoots of three independent transformants in an 18 hour 32p uptake assay at 2 mM phosphate; Bottom, a corresponding immunoblot performed on equal amounts of protein isolated from the ECM of three week-old wild-type Arabidopsis thaliana and the psNTP9 transgenics. Apyrase expressing plants also showed four times as much phosphatase activity in the extracellular matrix as the wild-type (FIG. 1C). (Note, OE 1 in the FIG. stands for over-expression 1 transgenic line).

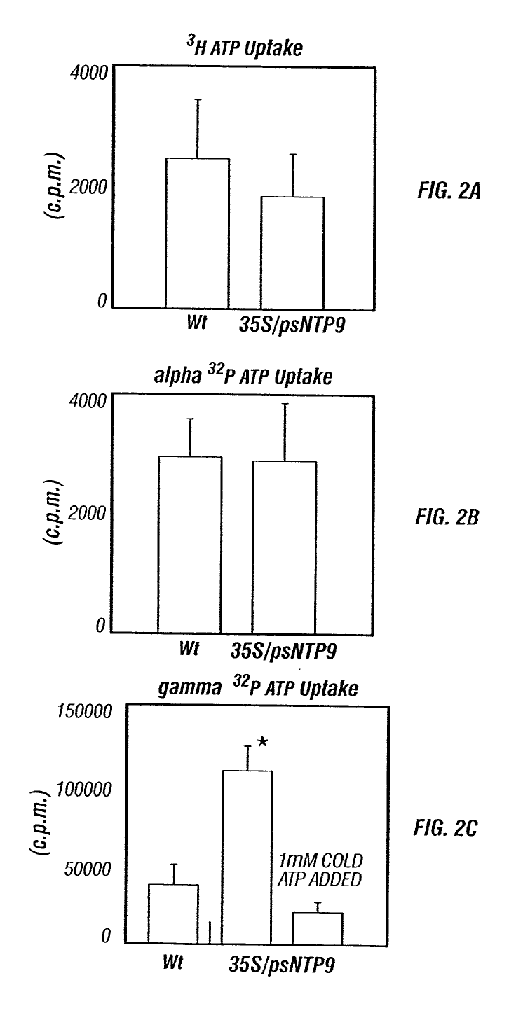

Transgenic plants preferentially transport the gamma phosphate of ATP: In order to address whether over-expression of ecto-apyrase was stimulating the adenosine salvage pathway, the intracellular uptake of adenosine was measured both in the presence and absence of the overexpression of apyrase. The inability of apyrase to translocate either extracellular AMP or adenosine was demonstrated by the low level of radiolabel accumulated in the transgenic plants fed [2,8³H]ATP and [a³²p]ATP (FIG. 2). The complete dephosphorylation of [2,8³H]ATP would result in a radiolabelled adenosine molecule while the complete dephosphorylation of [a³²P]ATP would result in a non-labeled adenosine label. FIG. 2A illustrates that plants overexpressing apyrase did not translocate radiolabelled adenosine (or byproducts of the dephosphorylation of [2,8³H]ATP) any more efficiently than plants not overexpressing apyrase (wild-type plants). FIG. 2B illustrates that plants overexpressing apyrase did not translocate AMP (or the byproducts of the dephosphorylated [a³²P]ATP) any more efficiently than wildtype plants. In comparison, feeding experiments where the y phosphate was labeled, the transgenics accumulated three times the amount of labeled phosphate as the wild-type (FIG. 2C). These data show that the over-expression of apyrase does not induce an increase in the uptake of adenosine and therefore its over-expression does not act to stimulate the adenosine salvage pathway.

### Example 4

### Ecto-Phosphatase is Involved in Drug Resistance in Yeast and Plants

### A. Materials and Methods

Expression of AtPGP-1 in yeast: The AtPGP-1 cDNA (Arabidopsis thaliana MDR gene, accession #X61370) was subcloned into pVT101 downstream of the ADH promoter to create the AtPGP-1/pVT101 construct. AtPGP-1/pVT101 and pVT101 were transformed into Saccharomyces cerevisiae INVSCI (genotype: MATa, his3-Δ1, leu2, trpl-289, ura3-52) and YMR4 (genotype: MATcris3-11,15, leu2-3, 112ura3Δ5, can Res pho5, 3: : ura3Δ1) by a PEG lithium acetate procedure (Eble, 1992) and selected on uracil dropout medium.

Yeast Growth: Yeast were grown at 30° C. under conditions of constant selection for uracil auxotrophy. YNB (Bio101, Vista, Calif.) supplemented with CSM (uracil dropout) and 2% glucose was used to grow strains having pVT101 constructs. Cycloheximide (Sigma Chemical, St. Louis, Mo.) was added to liquid media or spread on solid media to achieve a final concentration of 500 ng/ml. Nigencm (Sigma Chemical, St. Louis, Mo.) was added to liquid media or spread on solid media to achieve a final concentration of 25 μg/ml. Yeast strains used in cycloheximide selection assays were always propagated in the presence of the cycloheximide on plates and then streaked onto new plates containing drug or no drug, such that induced resistance existed in each strain at the time of the start of the assay. For selection assays on plates, single colonies were streaked; for selection in liquid media 0.01 ml of saturated culture was added to fresh media containing the drug. The plates shown in figures were grown for 3-5 days before photographs were taken. Yeast selection assays in liquid media were quantitated by turbidity as measured by absorbance at OD₆₀₀.

Expression of apyrase and AtPGP-1 in plants: The expression of apyrase in plants is as described above in Example 3. Similar methods were employed to express AtPGP-1 in Arabidopsis thaliana plants with the following modifications. The AtPGP-1 coding region was subcloned into a pBIN vector lacking the GUS gene as described in Sidler et al. (1998). This plasmid was then transformed into A. tumefaciens as described above, which was used to infect root calli to produce transgenic plants expressing AtPGP-1.

Plant growth: Arabidopsis thaliana seeds were sown in a solid germination media containing MS salt, 2% sucrose, 0.8% agar, and vitamins (Valvekens et al., 1992). For selection assays, cycloheximide was spread on the media to achieve a final concentration of 250 ng/ml. Plant growth was measured by germination percentage after 6-30 days.

### B. Results

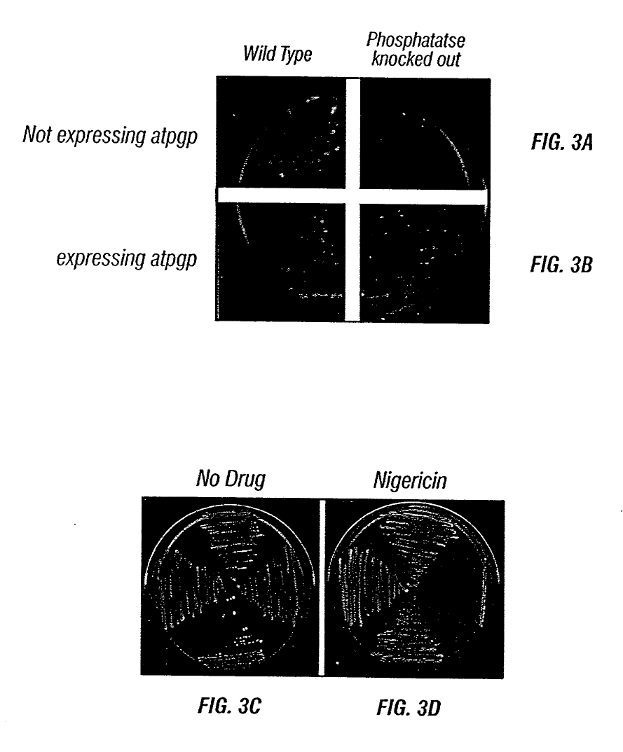

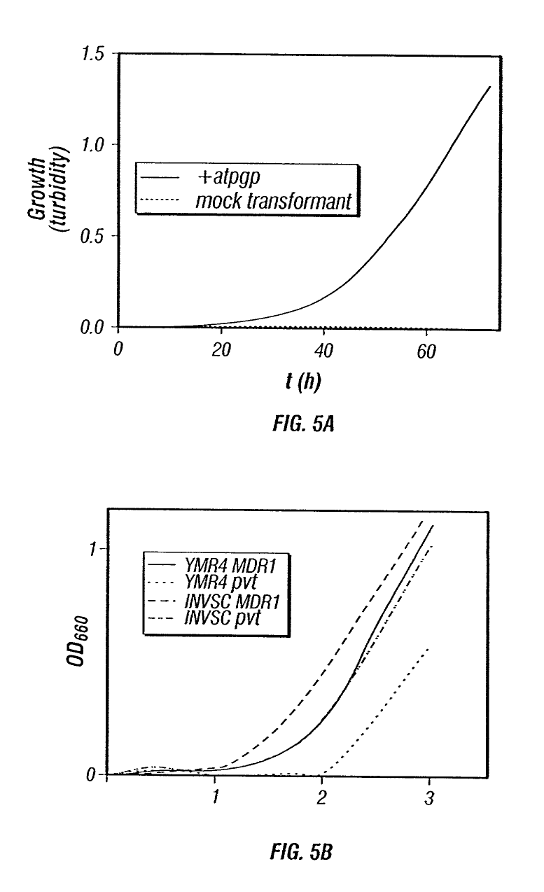

Effect of over-expression of AtPGP-1 in yeast: When a yeast mutant, YNIR4, which is deficient in two major extracellular phosphatases and tends to accumulate ATP extracellularly, was grown in a potent cellular toxin, cycloheximide, it did not grow whereas a wild-type yeast strain, INVSCI, did grow in the presence of cycloheximide (FIG. 3A). Surprisingly, expression of the plant multidrug resistance (MDR) gene, AtPGP-1, enabled the yeast mutant to grow in the toxin (FIG. 3B and FIG. 5A). The presence of AtPGP-1 in the wild-type yeast did not have any effect when grown in the presence of cycloheximide (FIG. 3B). The same result was obtained when the yeast strains were cultured in nigericin (FIG. 3C, 3D, FIG. 5B, 5C). In FIG. 3C and 3D, starting from the top of the dish clockwise, the cells are as follows: INVSCI (wild-type) overexpressing AtPGP-1, YNM4 containing the vector alone, Y4 overexpressing AtPGP-1, and INVSCI containing the vector alone. When grown without drug, all the cells grow (FIG. 3C). However, when grown in drug, only the YMR4 containing vector alone shows reduced growth. The survival of the AtPGP-1 transformed strains was due to the ability of the NIDR1 channel to efflux the toxin, hence lowering the actual cellular concentration of the poison cycloheximide. The sensitivity of the untransformed mutant to the drug is likely due to a loss of the ATP gradient below a point at which endogenous transporters, similar to AtPGP-1 can function.

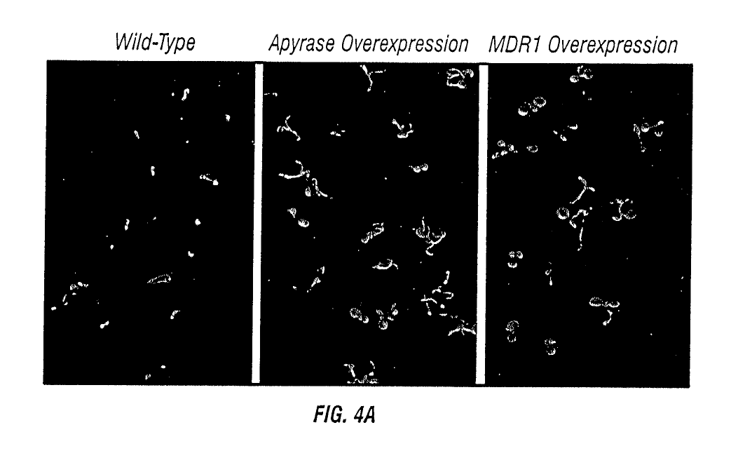

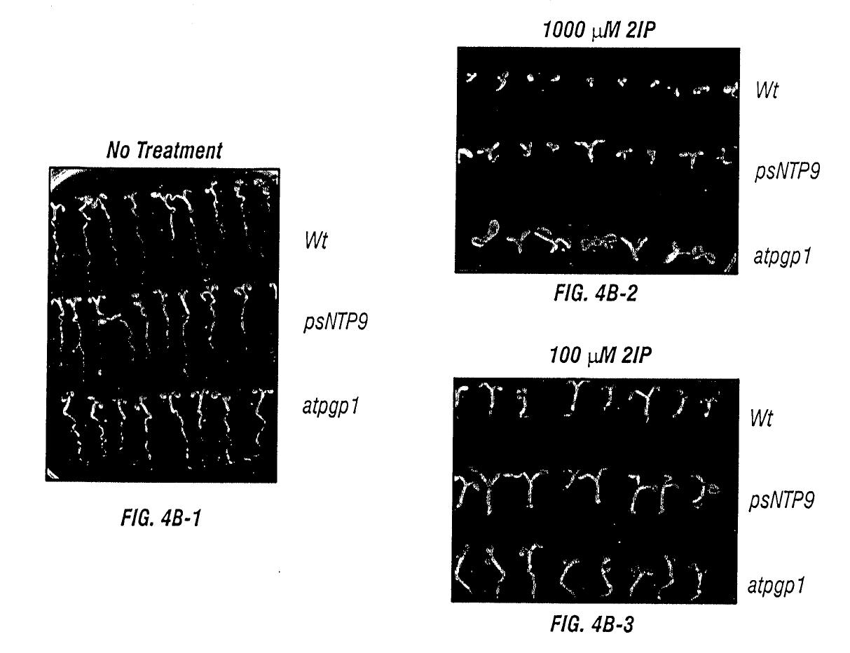

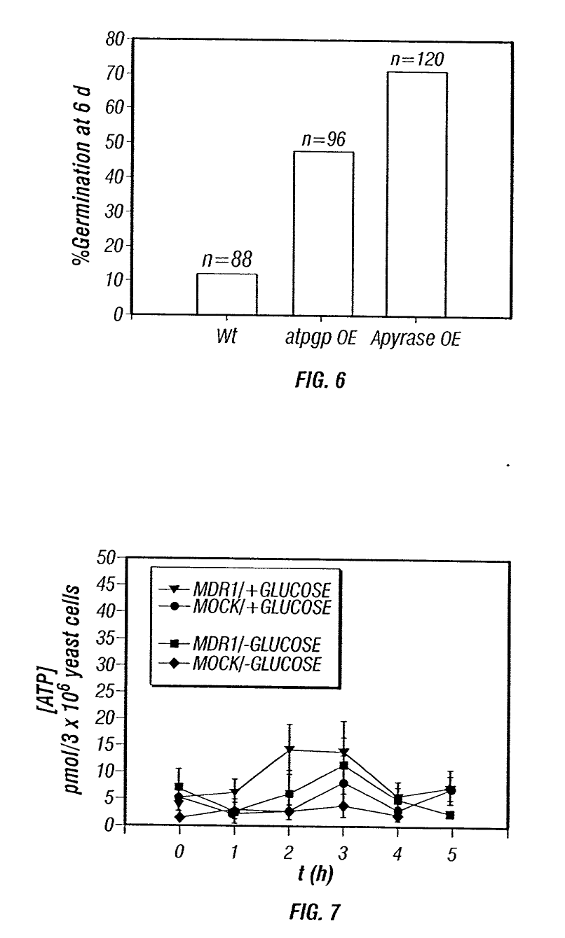

Effect of over-expression of AtPGP-1 in plants: The over-expression of AtPGP-1 was able to confer resistance to cycloheximide in plants (FIG. 4A and 6) and to the cytokinin, N₆-(2isopentenyl) adenine (21P) (FIG. 4B). These results had not been observed previously and in fact, the prior art actually teaches away from this finding suggesting that over-expression of plant AtPGP-1 is not involved in drug resistance (Sidler. et al., 1998). Therefore, this result was particularly unexpected in plants. Additionally, since Arabidopsis plants overexpressing AtPGP-1 are able to grow in both cycloheximide and cytokinin, this suggests that the conference of drug resistance by AtPGP-1 is likely to be seen with other chemicals as well and is not an isolated phenomenon.

Effect of over-expression of apyrase on drug resistance in plants: Another unexpected result was obtained when the plant apyrase gene was over-expressed in plants. Over-expression of apyrase in plants resulted in the conference of resistance to cycloheximide (FIG. 4A and 6). The same result was-obtained when the plants were grown in the presence of a cytokinin, N₆-(2isopentenyl) adenine (FIG. 4B). In fact, over-expression of apyrase is surprisingly able to raise the germination rate above the level obtained by the over-expression of the MDR gene AtPGP-1 (FIG. 4A, 4B and 6). Just as under-expression of phosphatase activity in a yeast mutant lacking two potent extracellular phosphatases diminished its resistance to cycloheximide (FIG. 3A), over-expression of a powerful extracellular ATP phosphatase in plants bolstered resistance. The fact that higher resistance was found in plants genetically manipulated only with respect to phosphatase over-expression and not MDR1, indicates that there likely exists other ATP-symporters used in detoxification in addition to MDR1. Minimally, the stronger ATP gradient set up by apyrase in the transgenic plants affects the kinetics of the wild-type MDRI.

### Example 5

### ATP Efflux in Yeast and Plants Overexpressing AtPGP-1

### A. Materials and Methods

ATP collection: Yeast cells used in the luciferase assays were grown for two days and then transferred to Lsh media at the time of the assay. From this time forward, the cells were kept at room temperature on a rotator. Every hour a 1 ml aliquot was taken, the cells in the aliquot were counted on a hemocytometer, a methylene blue viability assay was performed (Boyum and Guidotti, 1997), the cells were centrifuged, and the supernatant was stored in liquid nitrogen until all the aliquots were collected. For luciferase assays involving plants, Arabidopsis thaliana plants were grown in sterile culture at 22° C. under 150-200 pE of continuous light for at least 15 days. Foliar ATP was collected by placing a single 30 pl drop of luciferase buffer (Analytical Luminescence Laboratory, Cockeysville, Md.) on a leaf and, without making direct physical contact with the plant, the droplet was immediately collected and snap frozen. For each leaf, the area was approximated as an integrated area of a 2-D image of the leaf using NIH 1.52 software (Shareware, NIH).

Luminometry: Samples were reconstituted to a 100 pl final volume in Firelight™ buffer (Analytical Luminescence Laboratory, Cockeysville, Md.). After the buffer was added, all samples were kept on ice. ATP standards were reconstituted in 100 μl of Firelight™ buffer and the standards and sample were loaded into a 96-well plate and read en an automated Dynex Technologies Model MLX luminometer (Dynex Technologies, Chantilly, Va.). Samples, were processed with the addition of 50 μl of Firelight™ enzyme (Analytical Luminescence Laboratory, Cockeysville, Md.) followed by a reading delay of 1.0 second and an integration time of 10 seconds. Output was taken as an average for the integration time and then averaged for multiple samples. The sample handling time was less than 2 hours.

Pulse Chase experiments: Yeast were grown to saturation in liquid medium, as described above, centrifuged, and resuspended in fresh medium containing 1 μCi/ml ³H-adenosine (Amersham, Arlington Heights, Ill.). The cells were rotated at room temperature for 20 minutes to allow adenosine uptake. After 20 minutes the cells were centrifuged. The pellet was washed twice in ice cold medium, resuspended in culture medium at room temperature, divided equally between five types (five per cell line), and placed on a rotator. Every ten minutes a separate tube from each cell line was centrifuged and the pellet and supernatant were placed in separate scintillation vials. The efflux activity was expressed as the ratio of counts in the supernatant to counts in the pellet.

### B. Results

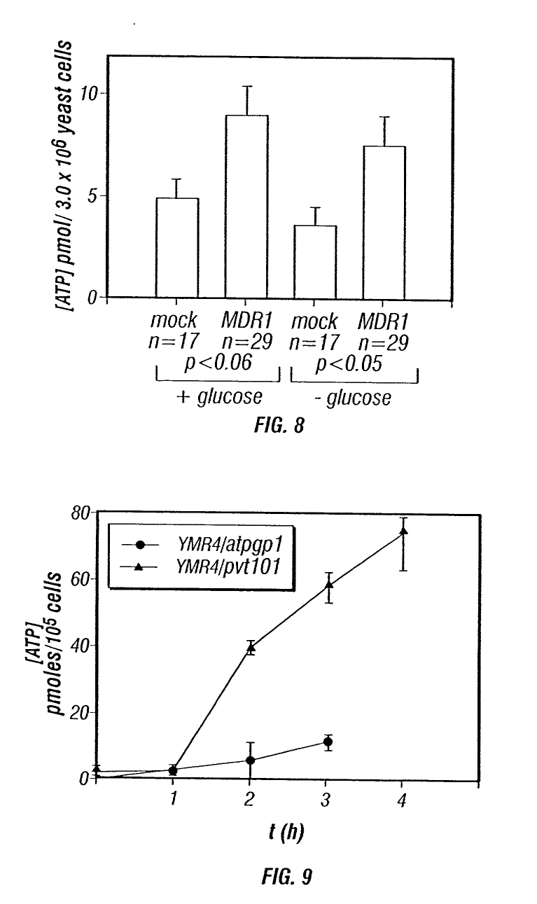

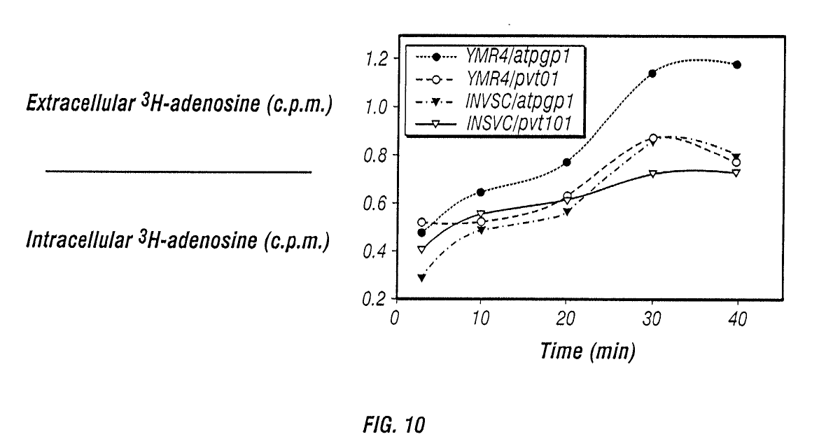

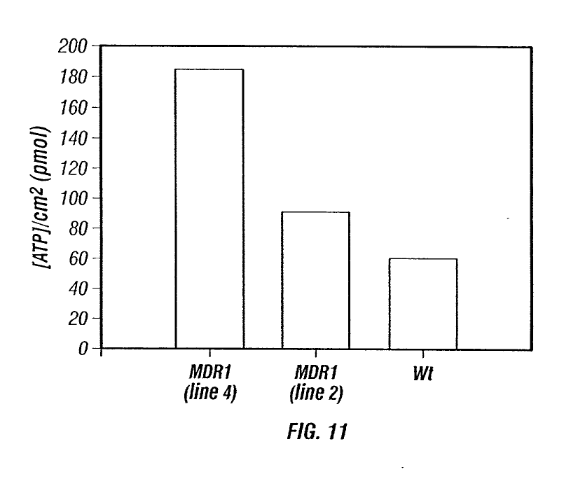

The ATP effluxed by the plant MDR1 AtPGP-1, over-expressed in yeast: In wild-type cells there is a steady-state level of ATP in the extracellular fluid, which is to say that the ATP outside the cells is rapidly degraded by phosphatases and does not accumulate over time (FIG. 7). However, the expression of the AtPGP-1 doubled this steady-state level (FIG. 8). If the yeast mutant, YMR4, which is deficient in extracellular phosphatase activity, is analyzed, there was a noticeable accumulation of ATP in the extracellular fluid compared to a control mutant transformed with empty plasmid pVT101 (FIG. 9). In addition to ATP measurements based on luminometry performed on a kinetic time-scale of hours, an earlier differential ATP efflux in MDRI expressing cells by pulse chase experiments was demonstrated (FIG. 10). Furthermore, Arabidopsis thaliana plants from two independently transformed lines, that constitutively express the AtPGP-1 protein, showed a significant accumulation of ATP on their leaf surfaces (FIG. 11). Taken together, these data demonstrate the absolute ability of plant MDRI, AtPGP1, to transport ATP from inside the cell to the outside. Moreover, these data show that ATP efflux channels and phosphatases both have roles in the steady-state level of ATP outside of the cell. This is the first demonstration of the importance of extracellular ATP steady-state levels, and the importance of an ATP gradient across biological membranes in the modulation of drug resistance.

### Example 6

### A Two-Component System is Found in Arabidopsis Plants

### A. Materials and Methods

Plant Growth: Arabidopsis seeds were sown in a solid germination media containing MS salts (Sigma Chemical, St. Louis, Mo.), 2% sucrose, 0.8% agar, and vitamins (Valvekens et al., 1992). For selection assays, one of the following, or a combination of both, was added to media (cooled to less than 50° C. before adding) immediately prior to pouring into plates: cycloheximide at a final concentration of 500 ng/ml;α, β-methyleneadenosine 5′-diphosphate at a final concentration of 1 mM. Plant growth was measured by germination percentage after 10-20 days.

All other materials and methods were discussed above in Example 4.

### B. Results

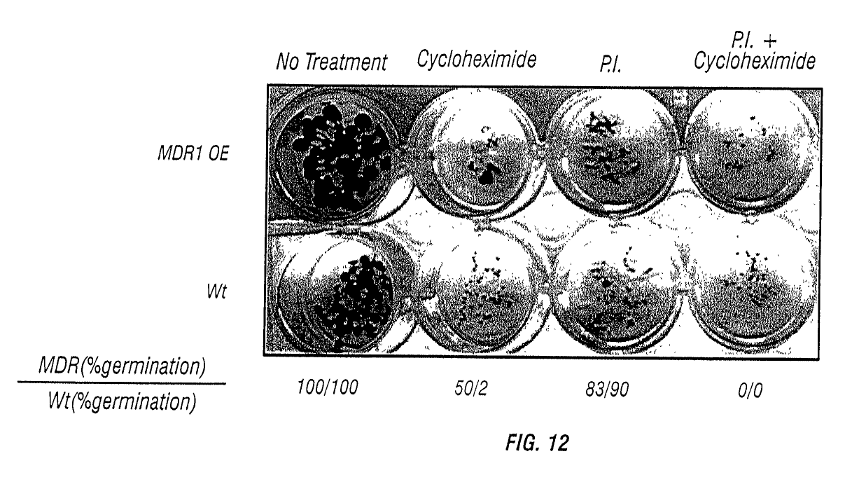

Effects of phosphatase inhibitor on plants overexpressing AtPGP-1: FIG. 12 shows that when wild-type and AtPGP-1 overexpressing (MDR OE) Arabidopsis thaliana plants were either treated with nothing (lane 1), cycloheximide (lane 2), a,(3-methyleneadenosine 5′-diphosphate (phosphatase inhibitor) (lane 3), or cycloheximide and phosphatase inhibitor (lane 4), both the wild-type and the AtPGP-1 overexpressing plants were affected similarly by the presence of phosphatase inhibitor. While the AtPGP-1 overexpressing plants grew significantly better in the presence of cycloheximide alone with a 50% germination rate for the AtPGP-1 overexpressing plants and a 2% germination rate for the wild-type plants, similar germination rates were seen for both the AtPGP-1 overexpressing and wild-type plants in the presence of either phosphatase inhibitor alone (83% and 90% germination respectively) or cycloheximide plus phosphatase inhibitor (no germination at all). The addition of phosphatase inhibitor surprisingly destroys the ability of the AtPGP-expressing plants to grow in the presence of cycloheximide. These data suggest that phosphatases are involved in the conference of drug resistance in plants and that there is a two-component system similar to that demonstrated in yeast in Example 4 and 5 above in which an MDR-like protein and an ATP-gradient-maintaining ecto-phosphatase are important in modulating drug resistance.

### Example 7

### The ATP Gradient Directly Effects Drug Resistance in Cells

### A. Materials and Methods

Cell lines: Cell lines were the same as those described above in Example 4 and 5. YMR4 MDRI is the phosphatase mutant yeast strain overexpressing AtPGP-1; YMR4 pVT101 contains vector alone; INVSC MLRI is the wild-type yeast strain overexpressing AtPGP-1; and INVSC pVT101 contains vector alone.

Selection in drug: To create drug resistant yeast strains, all four cell lines were grown up in the presence of 500 ng/ml of cycloheximide, and transferred to other cycloheximide containing plates after a period of four to six days. This transfer of cell lines and subculturing continued such that the yeast cells grew in the presence of cycloheximide for a period of at least a month. Cells cultured in media alone: To create cell lines that had not been preselected for their ability to grow in drug, yeast strains were grown on plates containing YNB (Bio101, Vista, Calif.) without uracil (-URA) to maintain the presence of the vector (which supplies UTRA) without any drugs added.

Growth of cells in suspension for ATP and drug selection experiments: Cells were transferred into 5 ml YNB -URA liquid media for turbidity measurements. All cell lines (both non-drug selected and drug-selected) were grown in media with the addition of either nothing, 500 ng/ml cycloheximide, 100 mM ATP, or 500 ng/ml cycloheximide and 100 mM ATP. Turbidity readings were taken after 48 hours.

Growth of cell lines in suspension for salvage pathway experiments: All cell lines were grown in liquid media either containing drug (for the drug selected lines) or not containing drug (for the non-drug selected lines). When the cultures reached a turbidity of 1.00 as measured at a wavelength of 600 in a spectrophotometer (OD₆₀₀=1.00), 10 μl of each culture was then removed and placed in either media with nothing added, 3 mM potassium phosphate; 3 mM adenosine; 9 mM potassium phosphate and 3 mM adenosine (for controls); potassium phosphate and cycloheximide; adenosine and cycloheximide; adenosine, cycloheximide, and potassium phosphate. Cell cultures were further grown for 72 hours, and their turbidity was determined by OD₆₀₀ readings on a spectrophotometer.

Growth of cell lines for nigericin experiments: Drug selected lines were removed from cycloheximide containing plates and placed in 5 ml liquid media containing 5 ng/ml cycloheximide. Cell cultures were allowed to grow until they reached an OD₆₀₀ reading of 1.00, and then 10 μl from each culture was removed and transferred to culture tubes containing 5 ml of liquid media and 25 pg/ml nigericin. OD600 readings were recorded daily for a period of up to 72 hours to determine growth.

### B. Results

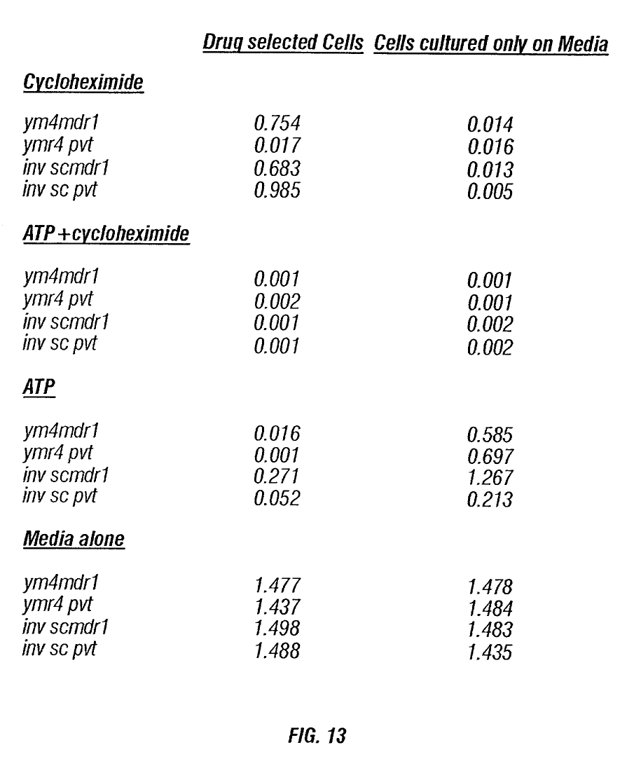

An ATP gradient is critical in MDR: The importance of the ATP gradient in MDR in yeast cells was demonstrated by showing that the growth of cells which were previously grown in drug and had developed resistance to the drug, were not able to grow in high levels of ATP unless they were overexpressing ATPGP-1 (FIG. 13). Cells which had not been previously selected in drug were able to grow in the presence of high levels of ATP (FIG. 13). These data emphasize that the loss of an ATP gradient is previously resistant cell lines abolishes resistance. This result is new to the understanding of MDR and has led to vast insight into the understanding of the mechanism by which MDR-ABC transporters confer resistance to cells and to methods to modulate such resistance. Moreover, when cells were grown in high levels of ATP and drug (cycloheximide), even the cell lines which had previously showed resistance to drug were unable to grow in the presence of drug and ATP. These data indicate that when the ATP gradient across biological membranes is destroyed (by the presence of high extracellular levels of ATP), efflux a of drugs cannot be achieved and therefore, drug resistance is abolished. In summary, the multidrug resistance channel is not functional without an ATP gradient.

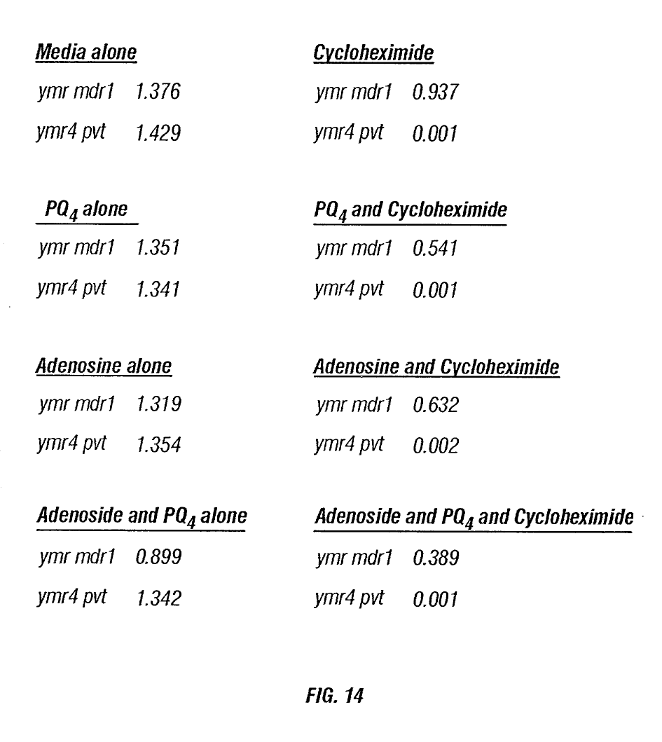

The drug resistance is not due to an adenosine salvage pathway: In order to address whether the involvement of a nucleotide salvage pathway was responsible for the results of the present invention, yeast cells were cultured in the presence of extracellular adenosine and extracellular phosphate. The acid phosphatase yeast mutant, YMR4, was selected because its decreased ectophosphatase activity makes it an ideal candidate for studying the effect of extracellular nucleotides on growth. If an adenosine salvage pathway were involved, then the presence of extracellular adenosine or possibly phosphate should help cells recoup the intracellular ATP losses due to ATP/drug efflux and should help cells grow in the presence of drug whether or not the cells were overexpressing AtPGP-1. In contrast, however, the addition of adenosine or phosphate to the media did not enhance resistance to the cells (FIG. 14). In fact, cells overexpressing AtPGP-1 grew best in drug alone, with the addition of adenosine and/or phosphate being slightly inhibitory. Furthermore, cells which did not express AtPGP-1 were unable to grow in drug regardless of the presence of adenosine and/or phosphate. These data suggest that an adenosine salvage pathway is not the principal mechanism at work in the present invention.

### Example 8

### High Throughput Screen for Isolating Apyrase Inhibitors

### A. Materials and Methods

Small Molecule Library: A small molecule library (DIVERSet format F), which was specifically constructed to maximize structural diversity in a relatively small library (9600 compounds), was obtained from ChemBridge Corporation (San Diego, Calif.). The small molecules (supplied in 0.1 mg dehydrated aliquots) were dissolved in DMSO, transferred to a 96 well plate, and tested for their ability to inhibit apyrase activity.

The assay: A stringent screen to test the ability of small molecules to disrupt the ATPase activity of the apyrase enzyme was developed based on phosphate-mobylate complexation. The assay was a modification of a phospholipase assay developed by Hergenrother et al. (1997): Under normal conditions, the apyrase enzyme liberates phosphate from ATP present in the reaction. The liberated phosphate quickly forms a complex upon addition of a small amount of acidified molybdate and ascorbate allowing for the production of a very dark blue color (the less phosphate liberated, the less blue color). Control reactions were performed with heat inactivated apyrase enzyme. Color intensity was detected on an Alpha Imager 2000 with AlphaEase™ software (Alpha Innotech, San Leandro, Calif.). Color changes were also evident by the naked eye. A Biomek 2000 robot (Beckman, Fullerton, Calif.) was used for screening the 9600 samples.

To each well of the 96 well plates containing a small molecule from the library, 100 pI of reaction buffer (60 mM HEPES, 3 MM MgCl₂, 3 mM CaCl₂, 3 mM ATP pH 7.0) was added. The apyrase (potato apyrase grade VI, Sigma Chemical, St. Louis, Mo.) enzyme (0.1 units) was added in a 5 pI volume and the reaction was allowed to proceed at room temperature for 60 minutes.

Three buffers were used to visualize activity: Buffer A: 2% Ammonium molybdate in water Buffer B: 1 I % Ascorbic acid in 37.5% aqueous TCA. Buffer C: 2% trisodium citrate, 2% acetic acid.

Immediately before developing the assay, buffers A and B were mixed in a 1:1 5 ratio. 50 pl of A:B was added to each well. The 96 well plate was then vibrated on a table surface to mix the solution. The deep blue color developed after approximately 2 minutes. After 2 minutes, 50 p.1 of buffer C was added to each well and the blue color became darker, increasing the sensitivity of the assay. The color intensified for up to one hour with no accompanying color change in the control wells containing heat inactivated apyrase enzyme. The color intensity for a single plate was measured on an Alpha Imager 2000 with AlphaEase™ software (Alpha Innotech, San Leandro, Calif.).

### B. Results:

Nineteen positives were identified from the 9600 compound DIVERSet library. Dose response assays revealed that fourteen showed weak inhibition, two showed medium inhibition (Formulas N and V), and three showed relatively strong inhibition (Formulas I, II and III).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,407,956

U.S. Pat. No. 4,880,918

U.S. Pat. No. 5,705,352

Abraham et al., Proc. Natl. Acad. Sci. USA, 90:312-316, 1993.

Al-Awgati, Science, 269:805-806, 1995.

Baker and Tenover, J. Clin. Microbiol., 34:2654-2659.1996.

Berry-Lowe et al., J. Molec. App. Gen., 1:483-498, 1982.

Binding, In: Plant Protoplasts, CRC Press, Boca Raton, 21-37, 1985.

Bissinger and Kucher, J. Biol. Chem., 269:4180-4186, 1994.

Boyum and Guidotti, Microbiology, 143:1901-1908, 1997.

Che, J. Biol. Chem., 267:9684-9688, 1992.

Dudler and Hertig, J. Biol. Chem., 267:5882-5888, 1992.

Eble, Biotechniques, 13:18-20, 1992.

Fromm et al, Proc. Natl. Acad. Sci. USA, 82:5824-5828, 1985.

Hergenrother et al., Lipids, 32:783788, 1997.

Higgins, Cell, 82:693-696, 1995.

Hooykass et al., In: Molecular Form and Function of the Plant Genome, Plenum Press, N.Y., 655-667, 1984.

Horsch et al., In: Current Communications in Molecular Biology, Cold Spring Harbor Press, Cold Spring Harbor, N.Y., 13-19, 1988.

Huang et al., Biochem. Biophys. Res. Commun., 182:836-843, 1992.

In: Current Protocols in Molecular Biology, John Wiley & Sons, Inc., Mass., USA (1998

Kolacskowski et al., J Biol. Chem., 271:31543-31548, 1996.

Maniatis et al., In: Molecular Cloning, A Laboratory Manual, Cold Spring Harbor Press, Cold Spring Harbor, N.Y., 1982.

Meyers, Encycl. Molec. Biol. Molec. Med., 6:125-133, 1997.

Paszkowski et al., EMBO J., 3:2717-2722, 1984.

PCT Application WO 98/46736

Plesner, Int. Rev. Cyto., 158:141-214, 1995.

Schardl et al., Gene, 61:1-11, 1987.

Schweibert, Cell, 81:1063-1073, 1995.

Sedaa et al., J. Pharmacol. Exp. Ther., 252:1060-1067, 1990.

Senior et al., FEBS Letters, 377:285-289, 1995.

Sidler et al., The Plant Cell, 10: 1623-1636, 1998.

Thomas et al., Plant Cell, 12:519-533, 2000.

Ujhazy et al., Int. J Cancer, 68:493-500, 1996.

Valvekens, Proc. Natl. Acad. Sci. USA, 85:5536-5540, 1992.

Van Veen etal., Nature, 391:291-295, 1998.

Wadkins and Roepe, Int. Rev. Cytolog, 171:121-165, 1997.

Wadkins and Roepe, Intl. Rev. Cytology, 171:121-165, 1997.

Wang and Guidotti, J. Biol. Chem., 271:9898-9901, 1996.

Weiner et al. J. Biol. Chem. 261:4529-4534, 1986.

Zimmerman, Biochem. J., 285:345-365, 1992.

What is claimed is:

1. A method of inhibiting the growth of a bacterial cell comprising contacting the cell with an ectophosphatase inhibitor.

2. The method of claim 1, wherein the ectophosphatase inhibitor is represented by formula XV or a pharmaceutical salt thereof:

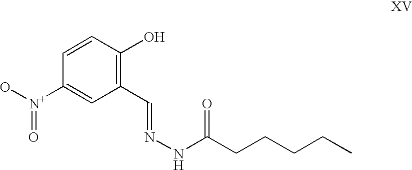

3. The method of claim 1, wherein the cell is an antibiotic resistant cell.

4. The method of claim 3, wherein the cell is resistant to methicillin.

5. The method of claim 4, wherein the cell is a Staphylococcus aureus cell.

6. The method of claim 1, further defined as a method of increasing the effectiveness of an antibiotic and further comprising contacting said cell with an antibiotic.

7. The method of claim 6, wherein the antibiotic is methicillin.

8. The method of claim 3, wherein said cell comprises an upregulated ecto-phosphatase relative to a bacteria that is not antibiotic resistant.

9. The method of claim 1, furtherer comprising contacting said cell with at least a second compound capable of inhibiting the growth of the cell.

10. The method of claim 1, wherein the compound is in a composition comprising a physiologically acceptable carrier or diluent.

11. The method of claim 10, wherein contacting comprises administering said composition to a mammal infected with said cell.

12. The method of claim 11, wherein the mammal is a human.

13. The method of claim 1, wherein the cell is from the family Staphylococcus.

14. The method of claim 13, wherein the cell is a Staphylococcus aureus cell.

15. A method of inhibiting the growth a tumor cell comprising contacting said cell with an ectophosphatase inhibitor.

16. The method of claim 15, wherein the cell is resistant to at least a first chemotherapeutic agent.

17. The method of claim 16, wherein the cell is resistant to vinblastine.

18. The method of claim 16, wherein said cell comprises an upregulated ecto-phosphatase relative to a tumor cell that is not resistant to said first chemotherapeutic agent.

19. The method of claim 15, further defined as a method of increasing the effectiveness of a chemotherapeutic agent and further comprising contacting said cell with at least a second compound capable of inhibiting the growth of the cell.

20. The method of claim 19, wherein the second compound is vinblastine.

21. The method of claim 15, wherein the ectophosphatase inhibitor is represented by formula VI or a pharmaceutical salt thereof:

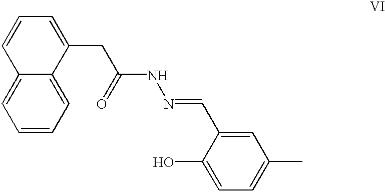

22. The method of claim 15, wherein the ectophosphatase inhibitor is represented by formula VIII or a pharmaceutical salt thereof:

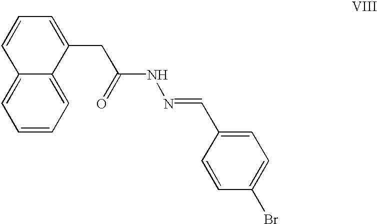

23. The method of claim 15, wherein the ectophosphatase inhibitor is represented by formula X or a pharmaceutical salt thereof:

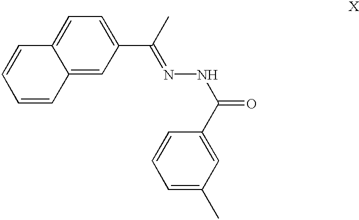

24. The method of claim 15, wherein the compound is in a composition comprising a physiologically acceptable carrier or diluent.

25. The method of claim 15, wherein contacting comprises administering said composition to a mammal with cancer.

26. The method of claim 25, wherein the mammal is a human.

27. The method of claim 15, wherein the cell is a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a bone cancer cell, a bone marrow cancer cell or a soft tissue cancer cell.

28. The method of claim 15, wherein the cell is a breast cancer cell.

29. A composition comprising, in a physiologically acceptable carrier or diluent, an antibiotic and a compound having formula XV or a pharmaceutical salt thereof:

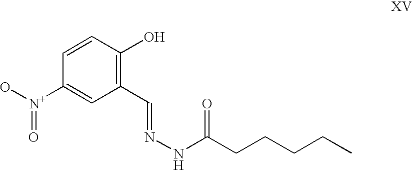

29. A composition comprising, in a physiologically acceptable carrier or diluent, a chemotherapeutic agent and a compound having formula VI:

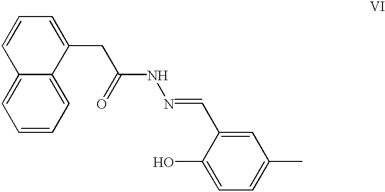

30. A composition comprising, in a physiologically acceptable carrier or diluent, a chemotherapeutic agent and a compound having formula VIII:

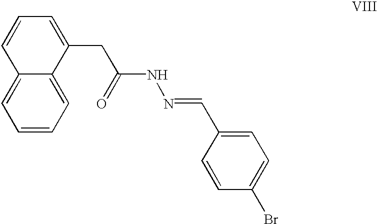

31. A composition comprising, in a physiologically acceptable carrier or diluent, a chemotherapeutic agent and a compound having formula X: [chemical structure]
